(19) United States
(12) Patent Application Publication
(10) Pub. No.: US 2022/0096537 A1
(43) Pub. Date: Mar. 31, 2022

(54) METHODS FOR TREATING DILATED CARDIOMYOPATHY

(71) Applicant: Mars, Incorporated, McLean, VA (US)

(72) Inventor: Anne Marie Bakke, Melton Mowbray (GB)

(73) Assignee: Mars, Incorporated, McLean, VA (US)

(21) Appl. No.: 17/482,985

(22) Filed: Sep. 23, 2021

Related U.S. Application Data
(60) Provisional application No. 63/083,492, filed on Sep. 25, 2020.

(51) Int. Cl. A61K 33/06; A61K 9/00; A61K 31/145; A61K 31/205; A61P 9/04; G01N 33/84; G01N 33/70; C12Q 1/42; G01N 33/80

(52) U.S. Cl. CPC A61K 33/06; A61K 9/0056; A61K 31/145; A61K 31/205; G01N 33/80; G01N 33/84; G01N 33/70; C12Q 1/42; A61P 9/04

(57) ABSTRACT

The presently disclosed subject matter relates to methods of determining the risk of and treating the development of dilated cardiomyopathy (DCM) and to methods of preventing and/or reducing a risk of developing DCM in a dog or canine. In certain embodiments, the method comprises determining biomarkers comprising hematocrit, phosphate, alkaline phosphatase or creatinine.

### CROSS-REFERENCE TO RELATED APPLICATIONS

This application claims priority to U.S. Provisional Application No. 63/083,492, filed on Sep. 25, 2020, the content of which is incorporated by reference in its entirety.

### TECHNICAL FIELD

The presently disclosed subject matter relates to methods for determining a pet's likelihood of developing dilated cardiomyopathy (DCM).

### BACKGROUND

Canine dilated cardiomyopathy (DCM) is a primary disease of cardiac muscle that results in a decreased ability of the heart to generate pressure to pump blood through the vascular system. Traditionally, dilated cardiomyopathy (DCM) is diagnosed when clinical signs become apparent as caused by cardiac dysfunction. These include signs such as panting, loss of energy, persistent cough, exercise intolerance and heart murmur, combined with a demonstration of structural and functional changes to the heart using imaging that indicates an enlarged heart with hypertrophic changes to the heart muscle's structure, and dysfunction, preferably using an ultrasound scan by a veterinary cardiologist. Radiographs can also be used to demonstrate an enlarged heart, but this will not provide evidence of dysfunction and is therefore not considered definitive.

Among causes, genetics plays a role with certain large breeds of dogs most predisposed to developing the condition as they age. However, taurine deficiency has also been demonstrated to cause DCM. Since 2018, certain diets have been associated with an increase in the prevalence of the condition in dogs across breed and age. These diets are typically so-called grain-free, legume-rich diets, especially those containing high inclusion levels of peas and/or lentils. Taurine deficiency does not appear to be the cause in most cases. More than 1000 cases have been reported to the FDA. The challenge is that by the time dogs show clinical signs, the heart's structural changes can have reached a point of heart failure, and the prognosis worsens. There is a need for methods that can help with early detection or diagnosis of DCM. There further is a need for providing treatment or customized recommendation to treat or to help to reduce the health risks associated with DCM.

### SUMMARY

The present disclosure provides a method of treating dilated cardiomyopathy (DCM) in a dog. In certain embodiments, the method comprises measuring, in a sample from the dog, an amount of at least one biomarker. In certain embodiments, the method comprises administering a treatment or a dietary regimen to treat or prevent DCM.

In certain embodiments, the at least one biomarker comprises hematocrit, inorganic phosphate, alkaline phosphatase, creatinine, or any combination thereof. In certain embodiments, an amount of the hematocrit is below about 45%. In certain embodiments, an amount of the inorganic phosphate is above about 1.5 mmol/L. In certain embodiments, an amount of the creatinine is below about 100 μmol/L. In certain embodiments, an amount of the alkaline phosphatase is above about 50 U/L.

In certain embodiments, the dog is at risk of developing DCM if an amount of hematocrit is below about 45%, an amount of the inorganic phosphate is above about 1.5 mmol/L, an amount of the creatinine is below about 100 μmol/L, and an amount of the alkaline phosphatase is above about 50 U/L.

In certain embodiments, the at least one biomarker further comprises a primary bile acid, a primary bile salt, a secondary bile acid, a secondary bile salt, alkaline phosphatase, amylase, total protein, BUN or urea level, phosphorus, calcium, urine protein, potassium, glucose, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine pH level, white blood cell count, or a combination thereof.

In certain embodiments, the dietary regimen comprises a low phosphorus diet, a low legume diet, a taurine supplement diet, a carnitine supplement diet, a low protein diet, a low sodium diet, a potassium supplement diet, a polyunsaturated fatty acid supplement diet, a liquid diet, a calcium supplement diet, a regular protein diet, or any combination thereof.

The present disclosure further provides a method of preventing or reducing the risk of developing dilated cardiomyopathy (DCM) in a dog. In certain embodiments, the method comprises measuring, in a sample from the dog, an amount of at least one biomarker. In certain embodiments, the method comprises administering a treatment or a dietary regimen to treat or prevent DCM.

In certain embodiments, the at least one biomarker comprise hematocrit, inorganic phosphate, alkaline phosphatase, creatinine, or any combination thereof. In certain embodiments, an amount of the hematocrit is below about 45%. In certain embodiments, an amount of the inorganic phosphate is above about 1.5 mmol/L. In certain embodiments, an amount of the creatinine is below about 100 μmol/L. In certain embodiments, an amount of the alkaline phosphatase is above about 50 U/L.

In certain embodiments, the dog is at risk of developing DCM if an amount of hematocrit is below about 45%, an amount of the inorganic phosphate is above about 1.5 mmol/L, an amount of the creatinine is below about 100 μmol/L, and an amount of the alkaline phosphatase is above about 50 U/L.

In certain embodiments, the at least one biomarker comprises a primary bile acid, a primary bile salt, a secondary bile acid, a secondary bile salt, alkaline phosphatase, amylase, total protein, BUN or urea level, phosphorus, calcium, urine protein, potassium, glucose, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine pH level, white blood cell count, or a combination thereof.

In certain embodiments, the dietary regimen comprises low legume diet, a taurine supplement diet, a carnitine supplement diet, a low phosphorus diet, a low protein diet, a low sodium diet, a potassium supplement diet, a polyunsaturated fatty acid supplement diet, a liquid diet, a calcium supplement diet, a regular protein diet, or any combination thereof.

### BRIEF DESCRIPTION OF THE DRAWINGS

FIG. 1 illustrates a chart of the pilot study described in the Example Section.

FIGS. 2A-2B illustrate hematological data from the pilot study. FIG. 2A shows red blood cell counts (RBC, ×10¹²/L) and FIG. 2B shows packed cell volumes (hematocrit, %, B) in Labrador retriever dogs fed either a mainstream control diet (blue) or a legume-rich test diet (green). Values are means and 95% confidence intervals indicated by the vertical lines. For blood samples analyzed at baseline (day 0), all dogs had been fed the control diet for 2.5 weeks, whereas samples analyzed from blood draws on days 3, 14 and 30 reflect the number of days that the test group dogs had been solely fed the test diet. Stars indicate a significant difference.

FIGS. 3A-3C illustrate biomarkers levels in Labrador retriever dogs fed either a mainstream control diet (left) or a legume-rich test diet (right). FIG. 3A shows plasma calcium levels (mmol/L). FIG. 3B shows whole blood ionized calcium levels (mmol/L). FIG. 3C shows plasma phosphorus (C) levels (mmol/L). Values are means and 95% confidence intervals indicated by the vertical lines. For blood samples analyzed at baseline (day 0), all dogs had been fed the control diet for 2.5 weeks, whereas samples analyzed from blood draws on days 3, 14 and 30 reflect the number of days that the test group dogs had been solely fed the test diet. Stars indicate a significant difference.

FIGS. 4A-4E illustrate levels of fecal biomarkers in Labrador retriever dogs fed either a main-stream control diet (left) or a legume-rich test diet (right). FIG. 4A shows fecal primary bile acids cholic acid (CA) levels (pmol/mg). FIG. 4B shows fecal chenodeoxycholic acid (CDCA) levels (pmol/mg). FIG. 4C shows fecal secondary bile acids deoxycholic acid (DCA) levels (pmol/mg). FIG. 4D shows fecal lithocholic acid (LCA) levels (pmol/mg). FIG. 4E shows fecal hyodeoxycholic acid (HDCA) levels (pmol/mg). Values are means and 95% confidence intervals indicated by the vertical lines. For blood samples analyzed at baseline (day 0), all dogs had been fed the control diet for 2.5 weeks, whereas samples analyzed from blood draws on days 4, 15 and 29 reflect the number of days that the test group dogs had been solely fed the test diet. Stars indicate a significant difference.

FIG. 5 shows the ranking of different analyzed features.

FIG. 6 shows a histogram correlating age and DCM relative probability of a DCM phenotype.

FIGS. 7A-7B show exploratory data analysis of hematocrit levels. FIG. 7A shows a normalized histogram of hematocrit. FIG. 7B shows the mean hematocrit levels and their correlation with data from the pilot study described herein.

FIGS. 8A-8B show exploratory data analysis of plasma phosphorus levels. FIG. 8A shows a normalized histogram of plasma phosphorus levels. FIG. 8B shows the mean phosphorus levels and their correlation with data from the pilot study described herein.

FIGS. 9A-9B show exploratory data analysis of blood alkaline phosphatase levels.

FIG. 9A shows a normalized histogram of alkaline phosphatase levels. FIG. 9B shows the mean alkaline phosphatase levels and their correlation with data from the pilot study described herein.

FIGS. 10A-10B show exploratory data analysis of blood creatinine levels. FIG. 10A shows a normalized histogram of creatine levels. FIG. 10B shows the mean creatine levels and their correlation with data from the pilot study described herein.

FIG. 11 shows an example of a machine learning diagnostic model.

FIGS. 12A-12E show the results of multi-group principle component analysis (PCA). The bi-plots A-E show the scores and loadings for each dataset generated from metabolomic profiling. FIG. 12A shows the PCA of fecal bile acids. FIG. 12B shows PCA of serum amino acids and biogenic amines. FIG. 12C shows PCA of lysophophatidylcholines (lysoPC).

FIG. 12D shows phosphatidylcholines (PCaa/ae). FIG. 12E shows hydroxy-sphingomyelins (SM[OH]) and sphingomyelins (SM). A positive association is indicated by lines in the same direction as the clusters while a negative association is indicated when lines move away from the clusters.

### DETAILED DESCRIPTION

The present disclosure is based, in part, on the discovery that specific biomarkers can be used for early diagnosis of DCM in dogs or canines. In certain embodiments, the biomarkers are biochemical markers. In certain embodiments, the biomarkers are demographic markers. In certain embodiments, the biomarkers comprise hematocrit, phosphate, alkaline phosphatase and creatinine.

For clarity and not by way of limitation, the detailed description of the presently disclosed subject matter is divided into the following subsections:

1. Definitions;

2. Biomarkers;

3. Test Methods;

4. Methods of Treatment; and

5. Devices and Systems.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition to reduce risk of DCM, and/or administering a composition to treat or delay the progression of DCM, an effective amount of a composition described herein is an amount sufficient to treat and/or ameliorate DCM, as well as decrease the symptoms and/or reduce the likelihood of developing DCM. An effective treatment described herein is a treatment sufficient to treat and/or ameliorate DCM, as well as decrease the symptoms and/or reduce the likelihood of DCM. The decrease can be a 10%, 20%, 30%, 40%, 50%, 60%, 70%, 80%, 90%, 95%, 98% or 99% decrease in severity of symptoms of DCM, or likelihood of DCM. An effective amount can be administered in one or more administrations. A likelihood of an effective treatment described herein is a probability of a treatment being effective, i.e., sufficient to treat and/or ameliorate DCM, as well as decrease the symptoms.

As used herein, and as well understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, reducing the likelihood of developing disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

The terms “animal” or “pet” as used in accordance with the present disclosure refers to domestic animals including, but not limited to, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like.

As used herein, the terms “dog” or “canine” are used interchangeably and refer to any member of the Canidae family including, but not limited to, Canis lupus, Canisfamiliaris, Canis latrans, Canis dingo, Lycaon pictus, Chrysocyon brachyurus, Atelocynus microis, Cuon alpinus, Speothos venaticus, Nyctereutes procyonoides, Vulpes vulpes, and Alopex lagopus. In certain embodiments, the dog or canine is Canisfamiliaris.

The term “pet food” or “pet food composition” or “pet food product” or “final pet food product” means a product or composition that is intended for consumption by, and provides certain nutritional benefit to a companion animal, such as a cat, a dog, a guinea pig, a rabbit, a bird or a horse. For example, but not by way of limitation, the companion animal can be a “domestic” dog or canine. A “pet food” or “pet food composition” or “pet food product” or “final pet food product” includes any food, feed, snack, food supplement, liquid, beverage, treat, toy (chewable and/or consumable toys), meal substitute or meal replacement.

The term “training dataset” means a database of unique dogs or canines that can be used to train the prediction model. In certain non-limiting embodiments, the “training dataset” can include demographic information, such as age, weight, breed, and reproductive status of the dog. The age can include the age of a pet during a visit to the veterinarian and/or the age of the pet during the first diagnosis of DCM. The breed can refer a homogeneous group of dogs having particular hereditary qualities. In certain non-limiting embodiments, the “training dataset” can include one or more biomarkers.

The term “visit” means a meeting between a healthcare practitioner or provider, such as a veterinarian, and a dog. In certain embodiments, a medical record is generated during or after a visit. In certain embodiments, an amount of one or more biomarkers is determined during a visit. In certain embodiments, a diagnosis of DCM is made during a visit. The practitioner can make a visit to the canine in a hospital and/or in a home or other location. A canine, taken by an owner, can make a visit to the practitioner in a clinic or an office.

The term “customized recommendation” means any treatment, method, or test used to lower/reduce the risk of developing DCM or reducing/managing the symptoms or effects of DCM. For example, the “customized recommendation” can include at least one of a therapeutic intervention, renal sparing strategy, or testing for disease progression.

### 2. Biomarkers

The present disclosure provides biomarkers and methods using the same to determine a susceptibility of a canine to develop DCM. In certain embodiments, one or more biomarkers or demographic information of a dog or canine can be used, in part, to determine a probability risk score for developing DCM.

The term “biomarker” means a characteristic that is objectively measured and evaluated as an indicator of normal biological processes, pathogenic processes, or pharmacologic responses to a therapeutic intervention. The term “biomarker” can also mean any substance, structure, or process that can be measured in the body or its products and influence or predict the incidence of outcome or disease. For example, the biomarker can be analyzed or determined from a urine or blood sample of a dog. For example, but without any limitation, the biomarker can include primary bile acids, primary bile salts, secondary bile acids, secondary bile salts, alkaline phosphatase, amylase, total protein, BUN or urea level, creatinine, phosphorus, calcium, urine protein, potassium, glucose, hematocrit, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine PH level, and/or white blood cell count. In certain embodiments, the one or more biomarkers can be obtained from the blood, urine, serum, plasma, saliva or feces of the dog or canine.

In certain embodiments, the one or more biomarker can be used for predicting DCM based on one or more biological parameters related to the development of DCM. In certain embodiments, the one or more biomarker can be used for customizing health recommendations. In certain embodiments, the health recommendation comprises a treatment disclosed herein.

In certain embodiments, the biomarker comprises the hematocrit level in the blood of the canine. In certain embodiments, the biomarker comprises the total creatinine level in the blood of the canine. In certain embodiments, the biomarker comprises the total creatinine level in the serum of the canine. In certain embodiments, the biomarker comprises the total creatinine level in the plasma of the canine. In certain embodiments, the biomarker comprises the inorganic phosphate level in the blood of the canine. In certain embodiments, the biomarker comprises the inorganic phosphate in the serum of the canine. In certain embodiments, the biomarker comprises the inorganic phosphate level in the plasma of the canine. In certain embodiments, the biomarker comprises the alkaline phosphatase level in the blood of the canine. In certain embodiments, the biomarker comprises the alkaline phosphatase level in the serum of the canine. In certain embodiments, the biomarker comprises the alkaline phosphatase level in the plasma of the canine. In certain embodiments, the biomarker comprises the level of primary bile acids and/or primary bile salts in the serum of the canine. In certain embodiments, the biomarker comprises the level of primary bile acids and/or primary bile salts in the feces of the canine. For example, but without any limitation, primary bile acids and/or salts include, e.g., cholic acid (CA), chenodeoxycholic acid (CDCA), α-muricholic acid (MCA[α]), β-muricholic acid (MCA[β]), taurocholic acid (TCA), taurochenodeoxycholic acid (TCDCA), α- and β-tauromuricholic acid (TMCA[α+β]), and salts thereof. In certain embodiments, the biomarker comprises the level of secondary bile acids and/or secondary bile salts in the serum of the canine.

In certain embodiments, the biomarker comprises the level of secondary bile acids and/or secondary bile salts in the feces of the canine. For example, but without any limitation, secondary bile acids and/or salts include, e.g., deoxycholic acid (DCA), lithocholic acid (LCA), hyodeoxycholic acid (HDCA), ursodeoxycholic acid (UDCA), taurodeoxycholic acid (TDCA), taurolithocholic acid (TLCA), glycodeoxycholic acid (GDCA), glycolithocholic acid (GLCA), and salts thereof. In certain embodiments, a change in a level of a biomarker is associated with an increased risk of developing DCM.

With each biomarker, an increased or a decreased level of the biomarker can give information about a dog or canine's susceptibility to developing DCM, depending on the particular biomarker. For example, in certain embodiments, a decreased level of hematocrit indicates an increased risk of developing DCM. In certain embodiments, an increased level of hematocrit indicates a decreased risk of developing DCM. In certain embodiments, a lower level of hematocrit compared to a predetermined reference value based on average levels of hematocrit in a control population of dogs or canines indicates an increased risk of developing DCM. In certain embodiments, a higher level of hematocrit compared to a predetermined reference value based on average levels of hematocrit in a control population indicates a decreased risk of developing DCM. In certain embodiments, the predetermined reference value of hematocrit is about 35%, about 37%, about 39%, about 41%, about 43%, about 45%, about 47%, about 49%, about 51%, about 53% or about 55%. In certain embodiments, the average levels of hematocrit in the control population is between about 37.3% and about 61.7%, between about 43% and about 61.7%, between about 47% and about 61.7%, between about 54% and about 61.7%, between about 58% and about 61.7%, between about 37.3% and about 43%, between about 37.3% and about 47%, between about 37.3% and about 54% or between about 58%.

In certain embodiments, an increased level of inorganic phosphate indicates an increased risk of developing DCM. In certain embodiments, a decreased level of inorganic phosphate indicates a decreased risk of developing DCM. In certain embodiments, a higher level of inorganic phosphate compared to a predetermined reference value based on average levels of inorganic phosphate in a control population of dogs or canines indicates an increased risk of developing DCM. In certain embodiments, a lower level of inorganic phosphate compared to a predetermined reference value based on average levels of inorganic phosphate in a control population indicates a decreased risk of developing DCM. In certain embodiments, the predetermined reference value of inorganic phosphate is about 1.4 mmol/l, about 1.5 mmol, about 1.6 mmol/l, about 1.7 mmol/l, about 1.8 mmol/l, about 1.9 mmol/l, about 2.0 mmol/l, about 2.5 mmol/l or about 3.0 mmol/l. In certain embodiments, the average levels of inorganic phosphate in the control population is between about 1.4 mmol/l and about 3 mmol/l, between about 1.5 mmol/l and about 3 mmol/l, between about 1.6 mmol/l and about 3 mmol/l, between about 1.7 mmol/l and about 3 mmol/l, between about 1.8 mmol/l and about 3 mmol/l, between about 1.9 mmol/l and about 3 mmol/l, between about 2.0 mmol/l and about 3 mmol/l or between about 2.5 mmol/l and about 3 mmol/l.

In certain embodiments, a decreased level of creatinine indicates an increased risk of developing DCM. In certain embodiments, an increased level of creatinine indicates a decreased risk of developing DCM. In certain embodiments, a lower level of creatinine compared to a predetermined reference value based on average levels of creatinine in a control population of dogs or canines indicates an increased risk of developing DCM. In certain embodiments, a higher level of creatinine compared to a predetermined reference value based on average levels of creatinine in a control population indicates a decreased risk of developing DCM. In certain embodiments, the predetermined reference value of creatinine is about 60 μmol/L, about 65 μmol/L, about 70 μmol/L, about 75 μmol/L, about 80 μmol/L, about 85 μmol/L, about 90 μmol/L, about 95 μmol/L or about 100 μmol/L. In certain embodiments, the average levels of creatinine in the control population is between about 60 μmol/L and about 120 μmol/L, about 65 μmol/L and about 120 μmol/L, about 70 μmol/L and about 120 μmol/L, about 75 μmol/L and about 120 μmol/L, about 80 μmol/L and about 120 μmol/L, about 85 μmol/L and about 120 μmol/L, about 90 μmol/L and about 120 μmol/L, about 95 μmol/L and about 120 μmol/L, about 100 μmol/L and about 120 μmol/L, about 110 μmol/L and about 120 μmol/L, about 60 μmol/L and about 65 μmol/L, about 60 μmol/L and about 70 μmol/L, about 60 μmol/L and about 75 μmol/L, about 60 μmol/L and about 80 μmol/L, about 60 μmol/L and about 85 μmol/L, about 60 pmol/L and about 90 μmol/L, about 60 μmol/L and about 95 μmol/L, about 60 μmol/L and about 100 μmol/L, or about 60 μmol/L and about 110 μmol/L.

In certain embodiments, an increased level of alkaline phosphatase indicates an increased risk of developing DCM. In certain embodiments, a decreased level of alkaline phosphatase indicates a decreased risk of developing DCM. In certain embodiments, a higher level of alkaline phosphatase compared to a predetermined reference value based on average levels of alkaline phosphatase in a control population of dogs or canines indicates an increased risk of developing DCM. In certain embodiments, a lower level of alkaline phosphatase compared to a predetermined reference value based on average levels of alkaline phosphatase in a control population indicates a decreased risk of developing DCM. In certain embodiments, the predetermined reference value of alkaline phosphatase is about 50 U/L, about 55 U/L, about 60 U/L, about 65 U/L, about 70 U/L, about 75 U/L, about 80 U/L, about 85 U/L, about 90 U/L, about 100 U/L, about 110 U/L, about 120 U/L, or about 130 U/L. In certain embodiments, the average levels of alkaline phosphate in the control population is between about 50 U/L and about 90 U/L, between about 60 U/L and about 90 U/L, between about 70 U/L and about 90 U/L, between about 80 U/L and about 90 U/L, between about 50 U/L and about 60 U/L, between about 50 U/L and about 70 U/L, between about 50 U/L and about 80 U/L, between about 60 U/L and about 70 U/L, or between about 70 U/L and about 80 U/L.

In certain embodiments, an increased level of primary bile acids and/or primary bile salts indicates an increased risk of developing DCM. In certain embodiments, a decreased level of primary bile acids and/or primary bile salts indicates a decreased risk of developing DCM. In certain embodiments, a higher level of primary bile acids and/or primary bile salts compared to a predetermined reference value based on average levels of primary bile acids and/or primary bile salts in a control population of dogs or canines indicates an increased risk of developing DCM. In certain embodiments, a lower level of primary bile acids and/or primary bile salts compared to a predetermined reference value based on average levels of primary bile acids and/or primary bile salts in a control population indicates a decreased risk of developing DCM.

In certain embodiments, a decreased level of secondary bile acids and/or secondary bile salts indicates an increased risk of developing DCM. In certain embodiments, an increased level of secondary bile acids and/or secondary bile salts indicates a decreased risk of developing DCM. In certain embodiments, a lower level of secondary bile acids and/or secondary bile salts compared to a predetermined reference value based on average levels of secondary bile acids and/or secondary bile salts in a control population of dogs or canines indicates an increased risk of developing DCM. In certain embodiments, a higher level of secondary bile acids and/or secondary bile salts compared to a predetermined reference value based on average levels of secondary bile acids and/or secondary bile salts in a control population indicates a decreased risk of developing DCM.

In certain embodiments, an increased or a decreased level of a biomarker can be detected in a current sample or in a recent medical record of a dog or canine (e.g., a record made within about 1 week, about 2 weeks, about 3 weeks, about 4 weeks, about 5 weeks, about 10 weeks, about 3 months or about 6 months before practicing any one of the methods disclosed herein). For example, but without any limitation, an increased or a decreased level of hematocrit can be found in a historic sample of the canine or in a historical medical record of the canine (e.g., a record made more than about 1 week, about 2 weeks, about 1 month, about 2 months, about 3 months or about 6 months before practicing any one of the methods disclosed herein).

In general, the ranges of average levels for the biomarkers can account for about 50% to about 100% of the healthy, normal population. For some biomarkers, the ranges of average levels for the biomarkers can account for about 80% to about 95%. Therefore, about 5% to about 25% of the population can have values above the higher end of an average/normal range, and about another about 5% to about 25% of the population can have values below the low end of an average/normal range. In certain embodiments, the actual ranges and validity of the biomarkers can be determined by each laboratory or testing, depending on the machine and/or on the population of canines tested to determine an average/normal range. Additionally, laboratory tests can be impacted by sample handling and machine maintenance/calibration. Updates to machines can also result in changes in the normal ranges. Any one of these factors can be considered for adjusting the average levels and/or the predetermined reference values of each biomarker.

In certain embodiments, the amounts of the biomarkers in the dog or canine can be detected and quantified by any means known in the art. For example, but without any limitation, automated analyzers, fluorescence-based assays, luminescence-based assays and/or antibody-based assays can be used.

In certain embodiments, other detection methods, such as other spectroscopic methods, chromatographic methods, labeling techniques, and/or quantitative chemical methods can be used. The level of a biomarker from a dog or canine and/or a predetermined reference value of the biomarker can be determined by the same method.

### 3. Test Methods

The present disclosure methods for determining susceptibility of a canine or dog to develop dilated cardiomyopathy (DCM) and methods of preventing and/or reducing a risk of a canine or dog of developing dilated cardiomyopathy (DCM). In certain embodiments, the method comprises: obtaining an amount of one or more biomarkers in the canine; and comparing the amount of each of the one or more biomarkers to a predetermined reference value. In certain embodiments, the predetermined reference value is based on an average amount of the biomarker in a sample in a control population. In certain embodiments, the one or more biomarkers comprises hematocrit, inorganic phosphate, creatinine, alkaline phosphatase, primary bile acids, primary bile salts, secondary bile acids, secondary bile salts, age, dog breed, or a combination thereof.

In certain embodiments, an amount of hematocrit below a first reference value indicates a risk of DCM. In certain embodiments, the first reference value is about 35%, about 37%, about 39%, about 41%, about 43%, about 45%, about 47%, about 49%, about 51%, about 53%, about 55%. In certain embodiments, the first reference value is between about 37.3% and about 61.7%, between about 43% and about 61.7%, between about 47% and about 61.7%, between about 54% and about 61.7%, between about 58% and about 61.7%, between about 37.3% and about 43%, between about 37.3% and about 47%, between about 37.3% and about 54% or between about 58%.

In certain embodiments, an amount of inorganic phosphate above a second reference value indicates a risk of DCM. In certain embodiments, the second reference value is above about 1.4 mmol/l, about 1.5 mmol, about 1.6 mmol/l, about 1.7 mmol/l, about 1.8 mmol/l, about 1.9 mmol/l, about 2.0 mmol/l, about 2.5 mmol/l or about 3.0 mmol/l.

In certain embodiments, an amount of creatinine below a third reference value indicates a risk of DCM. In certain embodiments, the third reference value is about 60 μmol/L, about 65 μmol/L, about 70 μmol/L, about 75 μmol/L, about 80 μmol/L, about 85 μmol/L, about 90 μmol/L, about 95 μmol/L or about 100 μmol/L. In certain embodiments, the third reference value is between about 60 μmol/L and about 120 μmol/L, about 65 μmol/L and about 120 μmol/L, about 70 μmol/L and about 120 μmol/L, about 75 μmol/L and about 120 μmol/L, about 80 μmol/L and about 120 μmol/L, about 85 μmol/L and about 120 μmol/L, about 90 μmol/L and about 120 μmol/L, about 95 μmol/L and about 120 μmol/L, about 100 μmol/L and about 120 μmol/L, about 110 μmol/L and about 120 μmol/L, about 60 μmol/L and about 65 μmol/L, about 60 μmol/L and about 70 μmol/L, about 60 μmol/L and about 75 μmol/L, about 60 μmol/L and about 80 μmol/L, about 60 μmol/L and about 85 μmol/L, about 60 μmol/L and about 90 μmol/L, about 60 μmol/L and about 95 μmol/L, about 60 μmol/L and about 100 μmol/L or about 60 μmol/L and about 110 μmol/L.

In certain embodiments, an amount of alkaline phosphatase above a fourth reference value indicates a risk of DCM. In certain embodiments, the fourth reference value is about 50 U/L, about 55 U/L, about 60 U/L, about 65 U/L, about 70 U/L, about 75 U/L, about 80 U/L, about 85 U/L, about 90 U/L, about 100 U/L, about 110 U/L, about 120 U/L, or about 130 U/L. In certain embodiments, the fourth reference value is between about 50 U/L and about 90 U/L, between about 60 U/L and about 90 U/L, between about 70 U/L and about 90 U/L, between about 80 U/L and about 90 U/L, between about 50 U/L and about 60 U/L, between about 50 U/L and about 70 U/L, between about 50 U/L and about 80 U/L, between about 60 U/L and about 70 U/L or between about 70 U/L and about 80 U/L.

In certain embodiments, the method of predicting a risk of dilated cardiomyopathy (DCM) for a canine comprises: receiving at least one input level of one or more biomarkers from samples taken from the canine; analyzing and transforming the at least one input level of the one or more biomarkers to derive a probability score or a classification label via a classification algorithm; and generating an output. In certain embodiments, the method of predicting a risk of dilated cardiomyopathy (DCM) for a canine comprises: receiving at least one input level of one or more biomarkers from samples taken from the canine and an input level of an age of the canine; analyzing and transforming the at least one input level of the one or more biomarkers and the input level of the age to derive a probability score or a classification label via a classification algorithm; and generating an output. In certain embodiments, the method of predicting a risk of dilated cardiomyopathy (DCM) for a canine comprises: receiving at least one input level of one or more biomarkers from samples taken from the canine, an input level of an age of the canine and an input of the breed of the canine; analyzing and transforming the at least one input level of the one or more biomarkers, the input level of the age and the input level of the breed to derive a probability score or a classification label via a classification algorithm; and generating an output. In certain embodiments, the method further comprises determining a health recommendation based on the determining or categorizing. In certain embodiments, the code, when executed by the processor, further causes the system to display the determination or categorization and customized recommendation on a graphical user interface. In certain embodiments, the age of the canine is the age when a method disclosed herein is carried out. In certain embodiments, the breed of the canine can increase the likelihood of developing DCM. For example, but without any limitation, the breed can be Afghan Hound, American Cocker Spaniel, Boxer, Dalmatian, Doberman Pinscher, English Bulldog, English Cocker Spaniel, Great Dane, Irish Wolfhound, Newfoundland, Saint Bernard and Scottish Deerhound.

In certain embodiments, the at least one of the one or more biomarkers comprises information relating to a hematocrit, inorganic phosphate, creatinine, alkaline phosphate, primary bile acids, primary bile salts, secondary bile acids, secondary bile salts, age, dog breed, or a combination thereof. In certain embodiments, the analyzing and transforming the at least one input level of the one or more biomarkers comprises organizing and modifying each input level. In certain embodiments, the at least one input level is normalized. In certain embodiments, the at least one input level is transformed into composite levels of one or more biomarkers. In certain embodiments, the at least one input level is transformed and/or adjusted according to biological information of the canine, e.g., weight, age, height, medical history, breed, etc. In certain embodiments, the at least one input level comprises sequential measurements of the one or more biomarkers measured at different time points.

In certain embodiments, the classification algorithm comprises code developed from a training dataset. In certain embodiments, the classification algorithm is developed using a machine learning technique, e.g., a training algorithm. In certain embodiments, the classification algorithm is a hard classifier that determines the classification label of whether the canine is at risk of developing DCM, which determines the probability score of the canine developing DCM.

In certain embodiments, the output is the classification label or the probability score. In certain embodiments, the step of obtaining the data comprises measuring an amount of each of the one or more biomarkers in a sample from the canine. In certain embodiments, the step of obtaining the data from the test sample comprises receiving the data from a third party that has measured an amount of each of the one or more biomarkers in a sample from the canine to determine the data. In certain embodiments, the sample from the individual is a blood sample.

In certain embodiments, the training dataset comprising medical information relating to both a first plurality of biomarkers from a first set of sample canines and a second plurality of biomarkers from a second set of sample canines. In certain embodiments, the first set of sample canines have been diagnosed with DCM and the second set of sample canines have not been diagnosed with DCM. In certain embodiments, the training dataset comprising amounts of the biomarkers from canines that have been diagnosed with DCM and canines that have not been diagnosed with DCM. In certain embodiments, the first plurality of biomarkers comprises at least one of a hematocrit level, a creatinine level, an inorganic phosphate level, an alkaline phosphatase level, or any combination thereof. In certain embodiments, the first plurality of biomarkers comprises any one of the biomarkers disclosed herein. In certain embodiments, the second plurality of biomarkers comprises at least one a hematocrit level, a creatinine level, an inorganic phosphate level, an alkaline phosphatase level, or any combination thereof. In certain embodiments, the second plurality of biomarkers comprises any one of the biomarkers disclosed herein.

In certain embodiments, if the data is classified as meaning a risk of DCM, the canine is predicted to have a greater likelihood of developing DCM as compared to if the data is classified as meaning a low risk of DCM.

In certain non-limiting embodiments, the method of determining susceptibility of a canine to developing dilated cardiomyopathy (DCM) comprises: obtaining data comprising amounts of a plurality of biomarkers in the canine and optionally an age of the canine; and performing an analysis on the data with an analytical algorithm, e.g., a classification algorithm, i.e., a classifier. In certain embodiments, the classification algorithm is developed by a machine learning algorithm. In certain embodiments, the classification algorithm is developed from a training dataset.

In certain non-limiting embodiments, a method of determining susceptibility of a canine to developing dilated cardiomyopathy (DCM) comprises: receiving at least one input level of one or more biomarkers from the canine, optionally receiving an input level of an age of the canine and/or an input level of a breed of the canine, wherein at least one of the one or more biomarkers comprises a hematocrit level, a creatinine level, an inorganic phosphate level, an alkaline phosphatase level, or any combination thereof, analyzing and transforming the at least one input level of the one or more biomarkers and optionally the input level of the age and/or breed, by organizing and/or modifying each input level to derive a probability score or a classification label via a classification algorithm, wherein the classification algorithm comprises code developed from a training dataset, the training dataset comprising medical information relating to a first plurality of biomarkers and optionally ages from a first set of sample canines and a second plurality of biomarkers and optionally ages from a second set of sample canines, wherein the classification algorithm is developed using a training algorithm; wherein the classification algorithm determines the classification label of whether the canine is at risk of developing DCM or determines the probability score of the canine developing DCM; generating an output, wherein the output is the classification label or the probability score; providing a customized recommendation, e.g., a dietary regimen and/or further monitoring the one or more biomarkers based on the output; and displaying the output and/or customized recommendation on a graphical user interface. In certain embodiments, the one or more biomarkers comprises information relating to a hematocrit level, a creatinine level, an inorganic phosphate level, an alkaline phosphatase level, primary bile acids, primary bile salts, secondary bile acids, secondary bile salts, age, breed, or any combination thereof.

In certain embodiments, the classification algorithm comprises an algorithm selected from: a logistic regression algorithm, an artificial neural network algorithm (ANN), a recurrent neural network algorithm (RNN), a K-nearest neighbor algorithm (KNN), a Naïve Bayes algorithm, a support vector machine algorithm (SVM), a random forest algorithm, an AdaBoost algorithm and any combination thereof. In certain embodiments, the classification algorithm comprises a regularization algorithm. In certain embodiments, a regularization algorithm prevents overfitting. In certain embodiments, the classification algorithm comprises a standard RNN algorithm comprising an input layer, an output layer and a hidden layer. In certain embodiments, the RNN comprises vanilla nodes and/or layers. In certain embodiments, the RNN comprises long short-term memory (LSTM) nodes and/or layers. In certain embodiments, the RNN comprises about 1, about 2, about 3, about 4, about 5, about 6, about 7, about 8, about 9, about 10 or more hidden layers. In certain embodiments, the RNN comprises between about 1 and about 3, between about 2 and about 4, between about 3 and about 5, between about 5 and about 10, between about 1 and about 4, between about 1 and about 5, or between about 2 and about 6 hidden layers.

In certain embodiments, each layer comprises at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 150, at least about 200, at least about 250, at least about 300, at least about 400, at least about 500 nodes, or any intermediate number or range of nodes. In certain embodiments, each layer comprises between about 2 and about 10, between about 2 and about 20, between about 3 and about 30, between about 2 and about 50, between about 3 and about 100, between about 4 and about 200, between about 5 and about 300, between about 10 and about 500, between about 2 and about 1000, between about 4 and about 500 nodes. In certain embodiments, each layer comprises between about 5 and about 300 nodes. In certain embodiments, each layer comprises between about 6 and about 250 nodes. In certain embodiments, each layer comprises between about 7 and about 200 nodes. In certain embodiments, a hidden layer comprises a tanh activation function.

In certain embodiments, the input levels of the biomarkers, the age and the breed of the canine relate to medical records of one or more visit of the canine. In certain embodiments, the input levels of the biomarkers, the age and the breed of the canine relate to medical records of at least about 2 visits, at least about 3 visits, at least about 4 visits, at least about 5 visits, at least about 6 visits, at least about 7 visits, at least about 8 visits, at least about 9 visits, at least about 10 visits or more of the canine. In certain embodiments, the input levels of the biomarkers, the age and the breed of the canine relate to medical records of between about 1 visit to about 10 visits, between about 2 visits to about 10 visits, between about 3 visits to about 10 visits, between about 1 visit to about 5 visits, between about 1 visit to about 3 visits, between about 2 visits to about 5 visits, between about 3 visits to about 5 visits of the canine.

In certain embodiments, the classification label or the probability score is transformed from a combination of intermediate probability scores, each of which is determined based on the input levels of the biomarkers, the age and the breed of the canine relating to a medical record of one visit of the canine. In certain embodiments, the classification label or the probability score relates to the canine's status of contracting dilated cardiomyopathy (DCM) at the time of the determination of the classification label or the probability score. In certain embodiments, the classification label or the probability score relates to the canine's risk of developing dilated cardiomyopathy (DCM) after the determination of the classification label or the probability score.

In certain embodiments, the classification label or the probability score relates to the canine's risk of developing dilated cardiomyopathy (DCM) about 1 month, about 2 months, about 3 months, about 4 months, about 5 months, about 6 months, about 7 months, about 8 months, about 9 months, about 10 months, about 11 months, about 12 months or more after the determination of the classification label or the probability score. In certain embodiments, the classification label or the probability score relates to the canine's risk of developing dilated cardiomyopathy (DCM) about 1 year, about 2 years, about 3 years, about 4 years, about 5 years or more after the determination of the classification label or the probability score. In certain embodiments, the classification label or the probability score relates to the canine's risk of developing dilated cardiomyopathy (DCM) between about 1 month and about 12 months, between about 1 month and about 6 months, between about 1 month and about 3 months, between about 3 months and about 12 months, between about 6 months and about 12 months, between about 3 months and about 6 months after the determination of the classification label or the probability score. In certain embodiments, the classification label or the probability score relates to the canine's risk of developing dilated cardiomyopathy (DCM) between about 1 year and about 5 years, between about 1 year and about 3 years, between about 1 year and about 2 years, between about 2 years and about 5 years, between about 2 years and about 3 years, between about 3 years and about 5 years after the determination of the classification label or the probability score.

In certain embodiments, the customized recommendation comprises diagnosing the presence of a comorbidity in the canine. In certain embodiments, the comorbidity is selected from the group consisting of hyperthyroidism, diabetes mellitus, hepatopathy, underweight, murmur, arthritis, malaise, constipation, gastroenteritis, vomiting, inflammatory bowel disease, crystalluria, enteritis, urinary tract infection, upper respiratory disease, urinary tract disease, obesity, inappropriate elimination, cystitis, colitis and any combination thereof. In certain embodiments, the comorbidity is selected from the group consisting of hyperthyroidism, diabetes mellitus, hepatopathy, underweight, murmur and any combination thereof.

### 4. Methods of Treating

The present disclosure provides methods of treating dilated cardiomyopathy for a canine. The present disclosure also provides methods of preventing or reducing the risk of developing dilated cardiomyopathy (DCM) for a canine. In certain embodiments, the method comprises administering a dietary regimen to treat or prevent DCM for a canine.

In certain embodiments, the canine is at risk of dilated cardiomyopathy. In certain embodiments, the canine is not known to be at risk of dilated cardiomyopathy. In certain embodiments, the canine has been diagnosed with dilated cardiomyopathy. In certain embodiments, the canine is not known to have dilated cardiomyopathy.

The present disclosure provides methods of treating, preventing and/or reducing a risk of developing dilated cardiomyopathy (DCM) for a canine, wherein the method comprises: determining whether the canine is at a risk of developing DCM using any of the biomarkers disclosed herein. In certain embodiments, the method comprises determining an amount of the one or more biomarkers in a sample from the canine. In certain embodiments, the one or more biomarkers comprises hematocrit, inorganic phosphate, alkaline phosphatase, creatinine, or any combination thereof. In certain embodiments, the method comprises determining whether the canine is at a risk of developing DCM using any of the prediction models disclosed herein.

In certain embodiments, the present disclosure provides methods of treating or preventing dilated cardiomyopathy (DCM) for a canine, wherein the method comprises: determining whether the canine is at a risk of developing DCM by using any of the biomarkers and/or prediction methods disclosed herein, wherein if the canine is determined to be at a risk of developing DCM, the method further comprises prescribing a treatment regimen to the canine. In certain embodiments, for example and without any limitation, the treatment regimen comprises a dietary therapy, a diuretic, an angiotensin converting enzyme (ACE) inhibitor, a cardiac glycoside, a vasodilator, a bronchodilator, a pimobendane, an anti-arrhythmic drug, or any combination thereof.

In certain embodiments, the treatment regimen is a dietary therapy. In certain embodiments, the dietary therapy comprises a diet selected from a low legume diet, a taurine supplement diet, a carnitine supplement diet, a low phosphorus diet, a low protein diet, a low sodium diet, a potassium supplement diet, a polyunsaturated fatty acid (PUFA, e.g., long chain omega-3 fatty acids) supplement diet, a liquid diet, a calcium supplement diet, a regular protein diet, and combinations thereof.

In certain embodiments, a low legume diet comprises between about 0.0001% and about 20%, between about 0.001% and about 10%, between about 0.01% and about 5%, between about 0.05% and about 2%, or between about 0.01% and about 1% legume on a weight by weight basis of a pet food. In certain embodiments, a low legume diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 10%, about 15%, about 20% protein, or any intermediate percentage or range of protein on a weight by weight basis of a pet food. In certain embodiments, a low legume diet comprises about 1 g/kg/day, about 2 g/kg/day, about 3 g/kg/day, about 4 g/kg/day, about 5 g/kg/day, about 6 g/kg/day, about 7 g/kg/day, about 8 g/kg/day, about 9 g/kg/day, about 10 g/kg/day, about 15 g/kg/day, about 20 g/kg/day or any intermediate amount or range of protein. In certain embodiments, a low legume diet comprises between about 1 g/kg/day and about 20 g/kg/day, between about 1 g/kg/day and about 50 g/kg/day, between about 2 g/kg/day and about 30 g/kg/day, between about 2 g/kg/day and about 10 g/kg/day, between about 2 g/kg/day and about 8 g/kg/day, between about 5 g/kg/day and about 20 g/kg/day or any intermediate amount or range of legume. In certain embodiments, a low legume diet comprises about 4 to about 6 g/kg/day or about 5 to about 5.5 g/kg/day.

In certain embodiments, a taurine supplement diet comprises between about 0.01% and about 5%, between about 0.1% and about 2%, between about 0.1% and about 1%, between about 0.05% and about 2%, or between about 0.5% and about 1.5% taurine on a weight by weight basis of a pet food. In certain embodiments, a taurine supplement diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% taurine, or any intermediate percentage or range of taurine on a weight by weight basis of a pet food. In certain embodiments, a taurine supplement diet comprises about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of taurine. In certain embodiments, a taurine supplement diet comprises between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of taurine. In certain embodiments, a taurine supplement diet comprises about 2 g/1000 kcal of taurine.

In certain embodiments, a carnitine supplement diet comprises between about 0.01% and about 5%, between about 0.1% and about 2%, between about 0.1% and about 1%, between about 0.05% and about 2%, or between about 0.5% and about 1.5% carnitine on a weight by weight basis of a pet food. In certain embodiments, a carnitine supplement diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% carnitine, or any intermediate percentage or range of carnitine on a weight by weight basis of a pet food. In certain embodiments, a carnitine supplement diet comprises about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of carnitine. In certain embodiments, a carnitine supplement diet comprises between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of carnitine. In certain embodiments, a carnitine supplement diet comprises about 2 g/1000 kcal of carnitine.

In certain embodiments, a low phosphorus diet comprises between about 0.01% and about 5%, between about 0.1% and about 2%, between about 0.1% and about 1%, between about 0.05% and about 2%, or between about 0.5% and about 1.5% phosphorus on a weight by weight basis of a pet food. In certain embodiments, a low phosphorus diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% phosphate, or any intermediate percentage or range of phosphate on a weight by weight basis of a pet food. In certain embodiments, a low phosphorus diet comprises about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of phosphate. In certain embodiments, a low phosphorus diet comprises between about 0.1 g/1000 kcal and about 0.5 g/1000 kcal, between about 0.5 g/1000 kcal and about 1.0 g/1000 kcal, between about 1.0 g/1000 kcal and about 2.0 g/1000 kcal, between about 2.0 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of phosphate. In certain embodiments, a low phosphorus diet comprises about 0.5% phosphate on a weight by weight basis of a pet food. (e.g., about 1.2 g/1000 kcal for the dry renal diet or about 1.0 g/1000 kcal for the wet renal diet). In certain embodiments, a low phosphorus diet comprises about 0.9 or 1% phosphate on a weight by weight basis of a pet food (e.g., about 1.8 g/1000 kcal for the dry maintenance diet or about 2.3 g/1000 kcal for the wet maintenance diet). In certain embodiments, a low phosphorus diet comprises between about 1.0 g/1000 kcal and about 1.5 g/1000 kcal of phosphorus. In certain embodiments, a low phosphorus diet comprises about 1.5 g/1000 kcal of phosphorus.

In certain embodiments, a calcium supplement diet comprises between about 0.01% and about 5%, between about 0.1% and about 2%, between about 0.1% and about 1%, between about 0.05% and about 2%, or between about 0.5% and about 1.5% calcium on a weight by weight basis of a pet food. In certain embodiments, a calcium supplement diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% calcium, or any intermediate percentage or range of calcium on a weight by weight basis of a pet food. In certain embodiments, a calcium supplement diet comprises about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of calcium. In certain embodiments, a calcium supplement diet comprises between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of calcium. In certain embodiments, a calcium supplement diet comprises about 2 g/1000 kcal of calcium.

In certain embodiments, a combinatory calcium supplement and low phosphorus diet comprises a calcium-phosphorus ratio (Ca:P ratio) of between about 1 and about 2, between about 1.1 and about 1.4, between about 1.2 and about 1.4, between about 1.1 and about 1.3, between about 1.3 and about 1.8, between about 1.4 and about 1.6, between about 1.5 and about 1.8, or between about 1.6 and about 1.8. In certain embodiments, a combinatory calcium supplement and low phosphorus diet comprises a calcium-phosphorus ratio (Ca:P ratio) of about 1, about 1.1, about 1.2, about 1.3, about 1.4, about 1.5, about 1.6, about 1.7, about 1.8, about 1.9, or about 2.0. In certain embodiments, a combinatory calcium supplement and low phosphorus diet comprises a calcium-phosphorus ratio (Ca:P ratio) of about 1.3.

In certain embodiments, a low sodium diet comprises between about 0.00001% and about 5%, between about 0.0001% and about 1%, between about 0.001% and about 0.1%, or between about 0.001% and about 0.05% sodium on a weight by weight basis of a pet food. In certain embodiments, a low sodium diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% sodium, or any intermediate percentage or range of sodium on a weight by weight basis of a pet food. In certain embodiments, a low sodium diet comprises about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day about 120 mg/kg/day, about 150 mg/kg/day, or any intermediate amount or range of sodium. In certain embodiments, a low sodium diet comprises between about 1 mg/1000 kcal and about 50 mg/1000 kcal, between about 2 mg/1000 kcal and about 20 mg/1000 kcal, between about 5 mg/1000 kcal and about 50 mg/1000 kcal, between about 1 mg/1000 kcal and about 10 mg/1000 kcal, between about 0.1 mg/1000 kcal and about 5 mg/1000 kcal, between about 0.1 mg/1000 kcal and about 10 mg/1000 kcal, between about 0.1 mg/1000 kcal and about 20 mg/1000 kcal, between about 0.1 mg/1000 kcal and about 40 mg/1000 kcal, between about 10 mg/1000 kcal and 20 mg/1000 kcal of sodium. In certain embodiments, a low sodium diet comprises about 0.4 to about 0.9 mmol/kg/day, or about 9.2 to about 20.7 mg/kg/day. In certain embodiments, a low sodium diet comprises about 2 mmol/kg/day or about 46 mg/kg/day.

In certain embodiments, a potassium supplement diet comprises between about 0.00001% and about 5%, between about 0.0001% and about 1%, between about 0.001% and about 0.1%, or between about 0.001% and about 0.05% potassium supplement on a weight by weight basis of a pet food in addition to the potassium existing in the pet food. In certain embodiments, a potassium supplement diet comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% or more potassium supplement on a weight by weight basis of a pet food in addition to the potassium existing in the pet food, or any intermediate percentage or range of potassium supplement in addition to the potassium existing in a pet food on a weight by weight basis of a pet food. In certain embodiments, a potassium supplement diet comprises about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day or more, or any intermediate amount or range of potassium supplement in addition to the potassium existing in a pet food. In certain embodiments, a potassium supplement diet comprises between about 1 mg/1000 kcal and about 10 mg/1000 kcal, between about 2 mg/1000 kcal and about 20 mg/1000 kcal, between about 5 mg/1000 kcal and about 50 mg/1000 kcal, between about 1 mg/1000 kcal and about 10 mg/1000 kcal, between about 0.1 mg/1000 kcal and about 5 mg/1000 kcal, between about 0.1 mg/1000 kcal and about 10 mg/1000 kcal, between about 0.1 mg/1000 kcal and about 20 mg/1000 kcal, between about 0.1 mg/1000 kcal and about 40 mg/1000 kcal, between about 10 mg/1000 kcal and 20 mg/1000 kcal of potassium supplement in addition to the potassium existing in a pet food.

In certain embodiments, a potassium supplement diet comprises between about 0.01% and about 5%, between about 0.1% and about 2%, between about 0.1% and about 1%, between about 0.05% and about 2%, or between about 0.5% and about 1.5% potassium on a weight by weight basis of a pet food. In certain embodiments, a potassium supplement diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5% potassium, or any intermediate percentage or range of potassium on a weight by weight basis of a pet food. In certain embodiments, a potassium supplement diet comprises about 0.1 g/1000 kcal, about 0.2 g/1000 kcal, about 0.3 g/1000 kcal, about 0.4 g/1000 kcal, about 0.5 g/1000 kcal, about 0.6 g/1000 kcal, about 0.7 g/1000 kcal, about 0.8 g/1000 kcal, about 0.9 g/1000 kcal, about 1.0 g/1000 kcal, about 1.1 g/1000 kcal, about 1.2 g/1000 kcal, about 1.3 g/1000 kcal, about 1.4 g/1000 kcal, about 1.5 g/1000 kcal, about 1.6 g/1000 kcal, about 1.7 g/1000 kcal, about 1.8 g/1000 kcal, about 1.9 g/1000 kcal, about 2.0 g/1000 kcal, about 2.1 g/1000 kcal, about 2.2 g/1000 kcal, about 2.5 g/1000 kcal, about 2.8 g/1000 kcal, about 3.0 g/1000 kcal, about 3.5 g/1000 kcal, about 4 g/1000 kcal, about 5 g/1000 kcal, about 10 g/1000 kcal, about 15 g/1000 kcal, about 20 g/1000 kcal, or any intermediate percentage or range of potassium. In certain embodiments, a potassium supplement diet comprises between about 0.1 g/1000 kcal and about 0.5 g/1000 kcal, between about 0.5 g/1000 kcal and about 1.0 g/1000 kcal, between about 1.0 g/1000 kcal and about 2.5 g/1000 kcal, between about 2.5 g/1000 kcal and about 5.0 g/1000 kcal, between about 0.01 g/1000 kcal and about 0.1 g/1000 kcal, between about 0.05 g/1000 kcal and about 1.0 g/1000 kcal, between about 0.1 g/1000 kcal and about 1 g/1000 kcal, between about 0.1 g/1000 kcal and about 2 g/1000 kcal, between about 1 g/1000 kcal and 2 g/1000 kcal of potassium. In certain embodiments, a potassium supplement diet comprises between about 2 g/1000 kcal and about 2.5 g/1000 kcal of potassium. In certain embodiments, a potassium supplement diet comprises about 2.1 g/1000 kcal of potassium.

In certain embodiments, a regular protein diet comprises a protein level of between about 70 g/1000 kcal and about 90 g/1000 kcal, between about 70 g/1000 kcal and about 75 g/1000 kcal, between about 70 g/1000 kcal and about 80 g/1000 kcal, between about 80 g/1000 kcal and about 90 g/1000 kcal, or between about 85 g/1000 kcal and about 90 g/1000 kcal. In certain embodiments, a regular protein diet comprises a protein level of about 73 g/1000 kcal, about 74 g/1000 kcal, or about 75 g/1000 kcal.

In certain embodiments, a low protein diet comprises between about 0.0001% and about 20%, between about 0.001% and about 10%, between about 0.01% and about 5%, between about 0.05% and about 2%, or between about 0.01% and about 1% protein on a weight by weight basis of a pet food. In certain embodiments, a low protein diet comprises about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 10%, about 15%, about 20% protein, or any intermediate percentage or range of protein on a weight by weight basis of a pet food. In certain embodiments, a low protein diet comprises about 1 g/kg/day, about 2 g/kg/day, about 3 g/kg/day, about 4 g/kg/day, about 5 g/kg/day, about 6 g/kg/day, about 7 g/kg/day, about 8 g/kg/day, about 9 g/kg/day, about 10 g/kg/day, about 15 g/kg/day, about 20 g/kg/day or any intermediate amount or range of protein. In certain embodiments, a low protein diet comprises between about 1 g/kg/day and about 20 g/kg/day, between about 1 g/kg/day and about 50 g/kg/day, between about 2 g/kg/day and about 30 g/kg/day, between about 2 g/kg/day and about 10 g/kg/day, between about 2 g/kg/day and about 8 g/kg/day, between about 5 g/kg/day and about 20 g/kg/day or any intermediate amount or range of protein. In certain embodiments, a low protein diet comprises about 4 to about 6 g/kg/day or about 5 to about 5.5 g/kg/day.

In certain embodiments, a PUFA supplement diet comprises between about 0.01% and about 30%, between about 0.1% and about 20%, between about 1% and about 10%, between about 0.1% and about 5%, or between about 1% and about 10% PUFA supplement in addition to the PUFA existing in a pet food on a weight by weight basis of a pet food. In certain embodiments, a PUFA supplement diet comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 10%, about 15%, about 20%, about 25%, about 30% or more PUFA supplement in addition to the PUFA existing in a pet food, or any intermediate percentage or range of PUFA supplement in addition to the PUFA existing in a pet food on a weight by weight basis of a pet food. In certain embodiments, a PUFA supplement diet comprises about 0.1 g/kg/day, about 0.5 g/kg/day, about 1 g/kg/day about 1 g/kg/day, about 2 g/kg/day, about 3 g/kg/day, about 4 g/kg/day, about 5 g/kg/day, about 6 g/kg/day, about 7 g/kg/day, about 8 g/kg/day, about 9 g/kg/day, about 10 g/kg/day, about 15 g/kg/day, about 20 g/kg/day, about 30 g/kg/day, about 40 g/kg/day, about 50 g/kg/day, about 60 g/kg/day, about 70 g/kg/day, about 80 g/kg/day, about 90 g/kg/day, about 100 g/kg/day or any intermediate amount or range of PUFA supplement in addition to the PUFA existing in a pet food. In certain embodiments, a PUFA supplement diet comprises between about 0.1 g/kg/day and about 20 g/kg/day, between about 1 g/kg/day and about 100 g/kg/day, between about 2 g/kg/day and about 200 g/kg/day, between about 5 g/kg/day and about 150 g/kg/day, between about 10 g/kg/day and about 100 g/kg/day, between about 5 g/kg/day and about 50 g/kg/day or any intermediate amount or range of PUFA supplement in addition to the PUFA existing in a pet food. In certain embodiments, a PUFA supplement diet comprises a PUFA level of between about 1 g/1000 kcal and about 10 g/1000 kcal, between about 1 g/1000 kcal and about 5 g/1000 kcal, between about 5 g/1000 kcal and about 10 g/1000 kcal, between about 1 g/1000 kcal and about 3 g/1000 kcal, between about 1 g/1000 kcal and about 2 g/1000 kcal, between about 2 g/1000 kcal and about 4 g/1000 kcal, between about 5 g/1000 kcal and about 8 g/1000 kcal, between about 7 g/1000 kcal and about 10 g/1000 kcal. In certain embodiments, a PUFA supplement diet comprises a PUFA level of about 1 g/1000 kcal, about 2 g/1000 kcal, about 3 g/1000 kcal, about 4 g/1000 kcal, about 5 g/1000 kcal, about 6 g/1000 kcal, about 7 g/1000 kcal, about 8 g/1000 kcal, about 9 g/1000 kcal, or about 10 g/1000 kcal. In certain embodiments, a PUFA supplement diet comprises a PUFA level of about 2 g/1000 kcal, or 2.1 g/1000 kcal.

In certain embodiments, a PUFA supplement diet comprises n-6 PUFA (e.g., plant oils). In certain embodiments, a PUFA supplement diet comprises n-3 PUFA (e.g., fish oils). In certain embodiments, a PUFA supplement diet comprises eicosapentaenoic acid (EPA) and/or docosahexaenoic acid (DHA).

In certain embodiments, the diet therapy includes a combination of the low legume diet, the taurine supplement diet, the carnitine supplement diet, the low phosphorus diet, the calcium supplement diet, the potassium supplement diet, and a regular protein diet.

In certain embodiments, the treatment regimen comprises a diuretic. Non-limiting examples of diuretic include furosemide, hydrochlorothiazide, cholothiazide, spironolactone, mannitol, dimethyl sulfoxide, thiazide diuretics, and potassium-sparing diuretics. In certain embodiments, the diuretic can be administered to a dog at a dose of about 0.05 mg/kg to about 100 mg/kg. In certain embodiments, a dog can be administered up to about 2,000 mg of the diuretic in a single dose or as a total daily dose. For example, but not by way of limitation, a dog can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the diuretic in a single dose or as a total daily dose. In certain embodiments, the dog can be administered from about 50 mg to about 1,000 mg of the diuretic in a single dose or a total daily dose.

In certain embodiments, the treatment regimen comprises an ACE inhibitor. In certain embodiments, the ACE inhibitor is enalapril. In certain embodiments, the ACE inhibitor is benazepril. In certain embodiments, the ACE inhibitor can be administered to a dog at a dose of about 0.05 mg/kg to about 100 mg/kg. In certain embodiments, a dog can be administered up to about 2,000 mg of the ACE inhibitor in a single dose or as a total daily dose. For example, but not by way of limitation, a dog can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the ACE inhibitor in a single dose or as a total daily dose. In certain embodiments, the dog can be administered from about 50 mg to about 1,000 mg of the ACE inhibitor in a single dose or a total daily dose. In certain embodiments, the treatment regimen comprises an inotrope. In certain embodiments, the inotrope is pimobendan. In certain embodiments, the inotrope is digoxin. In certain embodiments, the inotrope is dobutamine. In certain embodiments, the inotrope can be administered to a dog at a dose of about 0.05 mg/kg to about 100 mg/kg. In certain embodiments, a dog can be administered up to about 2,000 mg of the inotrope in a single dose or as a total daily dose. For example, but not by way of limitation, a dog can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the inotrope in a single dose or as a total daily dose. In certain embodiments, the dog can be administered from about 50 mg to about 1,000 mg of the inotrope in a single dose or a total daily dose.

In certain embodiments, the treatment regimen comprises a vasodilator. In certain embodiments, the vasodilator is nitroglycerin or nitroprusside. In certain embodiments, the vasodilator is amlodipine. In certain embodiments, the vasodilator is sildenafil. In certain embodiments, the vasodilator can be administered to a dog at a dose of about 0.05 mg/kg to about 100 mg/kg. In certain embodiments, a dog can be administered up to about 2,000 mg of the vasodilator in a single dose or as a total daily dose. For example, but not by way of limitation, a dog can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the vasodilator in a single dose or as a total daily dose. In certain embodiments, the dog can be administered from about 50 mg to about 1,000 mg of the vasodilator in a single dose or a total daily dose.

In certain embodiments, the treatment regimen comprises an antiarrhythmic. Non-limiting examples of antiarrhythmics include magnesium chloride, lidocaine, procainaimide, mexiletine, atenolol, esmolol, propranolol, carvedilol, sotalol, amiodatone, diltiazem regular, and ditiazem extended-release. In certain embodiments, the antiarrhythmic can be administered to a dog at a dose of about 0.05 mg/kg to about 100 mg/kg. In certain embodiments, a dog can be administered up to about 2,000 mg of the antiarrhythmic in a single dose or as a total daily dose. For example, but not by way of limitation, a dog can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the antiarrhythmic in a single dose or as a total daily dose. In certain embodiments, the dog can be administered from about 50 mg to about 1,000 mg of the antiarrhythmic in a single dose or a total daily dose.

### 5. Devices, Systems and Applications

The present disclosure also provides a device, a system and/or an application for the method(s) disclosed herein, e.g., for determining susceptibility or reducing a risk of developing DCM for a canine. The device, system and/or application enable a user, such as a caretaker or owner to evaluate the risk of developing DCM and take action by themselves, or with the aid of a healthcare professional/veterinarian to evaluate risk of developing DCM for a canine and administer suitable treatment to the canine, if needed.

In certain embodiments, a device is used to carry out the method(s) disclosed herein. In certain embodiments, the device is configured to accept a user input. In certain embodiments, the user input comprises levels of a plurality of biomarkers in the canine according to step of receiving input information, e.g., levels of one or more biomarkers, of a method disclosed herein. In certain embodiments, the plurality of biomarkers comprises hematocrit, creatinine, inorganic phosphate, alkaline phosphatase, or any combination thereof. In certain embodiments, the device automatically (or on request) performs an analysis and transformation step of a method disclosed herein, e.g., analyzing and transforming the input information of the one or more biomarkers to derive a probability score or a classification label. In certain embodiments, the analysis and transformation step is performed using a classification algorithm. The analysis provides a classification of a risk of developing DCM in the canine, and provides output information.

In certain embodiments, the device provides a message comprising a warning that the canine is determined as at risk of developing DCM. In certain embodiments, the results of the method(s) are provided by the device in a user interface. In certain embodiments, the device provides a recommendation of treatment/prevention suggestions according to a treatment/prevention method disclosed in the instant application, e.g., a diet and/or a dietary regime.

In certain embodiments, the device can be specially constructed for the required purposes, or it can comprise a general purpose computer selectively activated or reconfigured by a computer program/application stored in the computer. In certain embodiments, the computer program/application comprises code for carrying out any one of the methods disclosed herein. Such a computer program/application can be stored in a computer readable storage medium, such as, but is not limited to, read-only memories (ROMs), random access memories (RAMs), EPROMs, EEPROMs, flash memory, magnetic or optical cards, any type of disk including floppy disks, optical disks, CD-ROMs, and magnetic-optical disks, or any type of media suitable for storing electronic instructions, and each coupled to a computer system interconnect.

In certain embodiments, the device comprises a processor that executes an application that directs the device to provide data fields for entry of user input relating to a step of receiving input information and an analysis and transformation step. In certain embodiments, the application uses the processor to evaluate the risk of the canine developing DCM in certain period of time after a measurement of a biomarker. In certain embodiments, the application is an easily navigable application, e.g., online, to carry out any method(s) disclosed herein.

In certain embodiment, the device is a tablet, smartphone, desktop computer, laptop computer or personal digital assistant. In certain embodiment, the device is a mobile device, such as a smartphone and a tablet.

In certain embodiments, a system is also provided for the method(s) disclosed in the instant application, of determining whether a canine is at a risk of developing DCM. In certain embodiments, the system comprises a database connected to a remotely located device. In certain embodiments, the device comprises a processor executing an analysis that evaluates a determination according to the method(s) disclosed herein. In certain embodiments, the system and/or the device further comprises a communication device for transmitting and receiving information. In certain embodiments, at least one input level of a biomarker is received from a remote second system, via the communication device. In certain embodiments, the system and/or the device transmits the determination or categorization and customized recommendation to the remote second system, via the communication device.

Unless specifically stated otherwise as apparent from the following discussion, it is appreciated that throughout the description, discussions utilizing terms such as “processing” or “computing” or “calculating” or “determining” or “displaying” or “analyzing” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

The algorithms and displays presented herein are not inherently related to any particular computer or other device. Various general purpose systems can be used with the application in accordance with the teachings herein, or it can prove convenient to construct a more specialized device to perform the required method operations. The structure for a variety of these systems will appear from the description above. In addition, the present embodiments are not described with reference to any particular programming language, and various examples can thus be implemented using a variety of programming languages. All preferred features and/or embodiments of the methods and the diets/dietary regimes disclosed in the instant application apply to the device, the system and the application.

### EXAMPLES

The presently disclosed subject matter can be better understood by reference to the following. The below examples are exemplary only, and should in no way be taken as limiting.

### Example 1

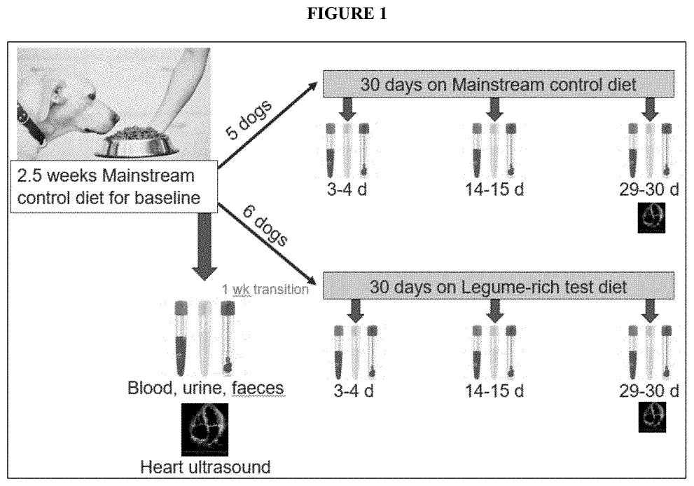

The present example provides novel biomarkers for identifying susceptibility to DCM. To this end, a pilot/proof of concept, longitudinal feeding trial was initiated to establish whether legume-containing diets lead to the development of DCM in dogs. A representative scheme of the trial is depicted in FIG. 1.

### Results

Two (2) different diets were used in the present example and were analyzed for their nutrient content (see Table 1).

TABLE 1
Nutrient analysis of the experimental diets as fed basis.

| Nutrients | Mainstream | Legume |
|---|---|---|
| per 1000 kcal | Control diet | Test diet |
| Proximate composition | | |
| Moisture, % | 7.9 | 9.8 |
| CHO, g | 136.41 | 130.14 |
| Protein, g | 65.34 | 69.42 |
| Fat, g | 27.45 | 30.70 |
| Fibre, g | 5.49 | 11.71 |
| Ash, g | 18.12 | 15.67 |
| Calculated ME, kcal/kg | 3643 | 3501 |
| Nutrient composition | | |
| Arginine, g | 3.60 | 4.77 |
| Histidine, g | 1.40 | 1.60 |
| Isoleucine, g | 2.31 | 2.68 |
| Leucine, g | 5.93 | 4.60 |
| Lysine, g | 3.02 | 4.34 |
| Methionine, g | 1.25 | 1.43 |
| Methionine + cystine, g | 2.04 | 2.06 |
| Phenylalanine, g | 2.83 | 3.00 |
| Phenylalanine + tyrosine, g | 4.80 | 4.80 |
| Taurine, g | 0.14 | 0.03 |
| Threonine, g | 2.31 | 2.46 |
| Tryptophan, g | 0.60 | 0.69 |
| Valine, g | 2.96 | 3.00 |
| Linoleic + Arachidonic acid, g | 7.58 | 11.85 |
| Linoleic acid, g | 7.44 | 11.80 |
| Arachidonic acid, g | 0.14 | 0.06 |
| Alpha linolenic acid, g | 0.38 | 4.66 |
| EPA + DHA, g | 0.11 | 0.11 |
| Calcium, g | 3.57 | 1.81 |
| Phosphorus, g | 2.50 | 1.39 |
| Ca:P ratio | 1.43 | 1.30 |
| Potassium, g | 1.81 | 3.21 |
| Sodium, g | 1.27 | 0.82 |
| Chloride, g | 2.39 | 2.43 |
| Magnesium, g | 0.32 | 0.37 |
| Copper, mg | 4.57 | 6.26 |
| Iodine, mg | 0.19 | 0.60 |
| Iron, mg | 26.6 | 56.3 |
| Manganese, mg | 3.52 | 9.03 |
| Selenium, μg | 175.7 | 322.8 |
| Zinc, mg | 65.9 | 101.4 |
| Vitamin A, IU | 1641.7 | 4284.5 |
| Vitamin D, IU | 257.2 | 464.0 |
| Vitamin E, IU | 51.06 | 35.13 |
| Thiamine, mg | 2.05 | 4.11 |
| Riboflavin, mg | 12.08 | 18.02 |
| Pantothenic acid, mg | 13.31 | 31.42 |
| Pyridoxine, mg | 10.84 | 17.77 |
| Vitamin B12, μg | 15.54 | 31.71 |
| Niacin, mg | 81.26 | 107.40 |
| Folic acid, μg | 204.5 | 262.8 |
| Biotin, μg | 576.5 | 991.1 |
| Choline, mg | 475.8 | 1028.6 |
| Vitamin K, mg | — | 0.29 |

Both diets were AAFCO, NRC and FEDIAF compliant, but differed in nutrient levels. The control diet was higher in carbohydrates and ash, while the test diet was higher in protein, fat and contained twice as much fiber. Of note, the control diet was higher in leucine, taurine, calcium, phosphorus and vitamin A, while the test diet contained higher lysine, linoleic acid, alpha-linolenic acid, potassium, microminerals and most vitamins. Both diets were palatable and well-accepted by the dogs during the trial, with no reports of meal refusals or adverse effects on fecal quality. Fecal quality remained within an acceptable range, however, the dogs fed the legume-rich Test diet produced higher quantities following 30 days on the diet.

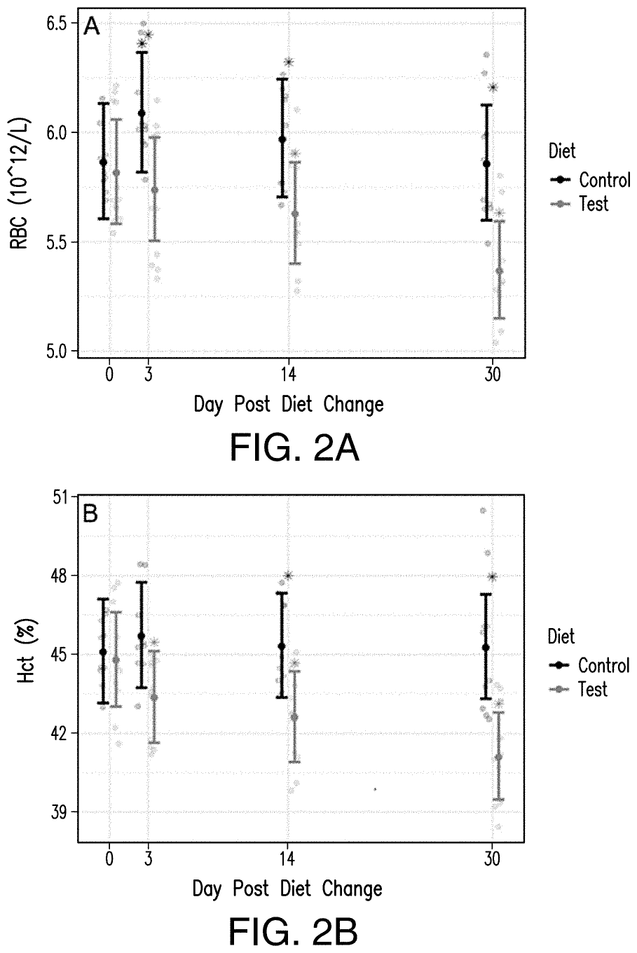

Blood samples were collected and hematological analysis was performed. Red blood cell counts (FIG. 2A) were significantly reduced compared to baseline in dogs fed the test diet on days 14 and 30, with five and six of six dogs on the test diet demonstrating counts below the lower limit of the reference range (6-1012/L), at days 14 and 30, respectively. Thus, the dogs were considered anemic. The concomitant reduction in hematocrit (FIG. 2B) support the reduced red blood cell numbers, while the red blood cell mean cell volume (MCV) and distribution width (RDW; Table 2) did not change consistently from baseline in the test dogs, indicating that the number more than the size of the red blood cells changed during the trial. Thus, the anemia in the test dogs would be considered normocytic.

TABLE 2
Mean hematology values with 95% confidence intervals for each diet group (Control, n = 5;
Test, n = 6) and sampling time (baseline and 3, 14 and 30 days), as well as results of
the statistical analysis (p-values). Lab reference ranges for each parameter are provided in italics.

| | Sampling | Control | | Test | | Statistical analysis | |
|---|---|---|---|---|---|---|---|
| Parameter | time | Mean | 95% CI | Mean | 95% CI | Diff. within | Diff. bet- |
| RBC (10¹²/L) | Baseline | 5.86 | (5.60, 6.13) | 5.82 | (5.58, 6.06) | — | — |
| 6-9 | Day 3 | 6.09* | (5.82, 6.37) | 5.74 | (5.50, 5.98) | NS | 0.034 |
| | Day 14 | 5.97 | (5.71, 6.24) | 5.63* | (5.40, 5.86) | 0.049 | 0.036 |
| | Day 30 | 5.86 | (5.60, 6.13) | 5.37* | (5.15, 5.59) | <0.001 | <0.001 |
| Haemoglobin (g/dL) | Baseline | 15.9 | (15.2, 16.7) | 15.8 | (15.1, 16.4) | — | — |
| 12-19 | Day 3 | 16.6* | (15.8, 17.3) | 15.8 | (15.1, 16.5) | NS | NS |
| | Day 14 | 16.2 | (15.5, 17.0) | 15.3 | (14.7, 15.9) | NS | NS |
| | Day 30 | 16.4 | (15.7, 17.2) | 14.8* | (14.2, 15.4) | <0.001 | <0.001 |
| Haematocrit (%) | Baseline | 45.1 | (43.1, 47.1) | 44.8 | (43.0, 46.6) | — | — |
| 40-55 | Day 3 | 45.7 | (43.7, 47.7) | 43.4* | (41.6, 45.1) | 0.035 | NS |
| | Day 14 | 45.3 | (43.4, 47.3) | 42.6* | (40.9, 44.3) | <0.001 | 0.011 |
| | Day 30 | 45.3 | (43.3, 47.3) | 41.1* | (39.5, 42.8) | <0.001 | <0.001 |
| MCV (fL) | Baseline | 76.9 | (75.4, 78.4) | 77.0 | (75.6, 78.4) | — | — |
| 60-77 | Day 3 | 75.1* | (73.6, 76.6) | 75.6* | (74.2, 77.0) | <0.001 | NS |
| | Day 14 | 75.9* | (74.4, 77.4) | 75.7* | (74.3, 77.1) | <0.001 | NS |
| | Day 30 | 77.3 | (75.8, 78.8) | 76.6 | (75.2, 78.0) | NS | NS |
| MCH (pg) | Baseline | 27.2 | (26.5, 27.9) | 27.1 | (26.5, 27.7) | — | — |
| 17-23 | Day 3 | 27.2 | (26.6, 27.9) | 27.5 | (26.9, 28.2) | NS | NS |
| | Day 14 | 27.2 | (26.5, 27.8) | 27.2 | (26.6, 27.8) | NS | NS |
| | Day 30 | 28.0* | (27.3, 28.7) | 27.5 | (26.9, 28.1) | NS | NS |
| MCHC (g/dL) | Baseline | 35.4 | (34.7, 36.0) | 35.2 | (34.6, 35.8) | — | — |
| 31-34 | Day 3 | 36.2* | (35.6, 36.9) | 36.4* | (35.9, 37.0) | <0.001 | NS |
| | Day 14 | 35.8 | (35.2, 36.4) | 35.9* | (35.4, 36.5) | 0.023 | NS |
| | Day 30 | 36.2* | (35.6, 36.8) | 35.9* | (35.4, 36.5) | 0.021 | NS |
| RDW (%) | Baseline | 13.3 | (12.7, 13.9) | 13.7 | (13.1, 14.3) | — | — |
| 14-17 | Day 3 | 14.0* | (13.3, 14.6) | 13.6 | (13.0, 14.2) | NS | 0.003 |
| | Day 14 | 14.4* | (13.8, 15.1) | 13.8 | (13.2, 14.3) | NS | <0.001 |
| | Day 30 | 14.2* | (13.6, 14.9) | 13.6 | (13.0, 14.2) | NS | <0.001 |
| WBC (10⁹/L) | Baseline | 5.40 | (4.25, 6.85) | 5.41 | (4.35, 6.72) | — | — |
| 6-12 | Day 3 | 6.28* | (4.95, 7.97) | 5.35 | (4.30, 6.65) | NS | 0.049 |
| | Day 14 | 5.85 | (4.61, 7.43) | 5.79 | (4.66, 7.20) | NS | NS |
| | Day 30 | 6.01 | (4.73, 7.63) | 5.53 | (4.45, 6.87) | NS | NS |
| Lymphocytes | Baseline | 1.44 | (1.17, 1.71) | 1.36 | (1.11, 1.61) | — | — |
| (10⁹/L) | Day 3 | 1.60 | (1.33, 1.87) | 1.46 | (1.21, 1.71) | NS | NS |
| 1.2-3.2 | Day 14 | 1.41 | (1.14, 1.68) | 1.43 | (1.18, 1.68) | NS | NS |
| | Day 30 | 1.66 | (1.39, 1.93) | 1.42 | (1.17, 1.67) | NS | NS |
| Monocytes (10⁹/L) | Baseline | 0.55 | (0.43, 0.67) | 0.53 | (0.42, 0.64) | — | — |
| 0.3-0.8 | Day 3 | 0.61 | (0.49, 0.73) | 0.60* | (0.49, 0.71) | 0.044 | NS |
| | Day 14 | 0.60 | (0.48, 0.72) | 0.57 | (0.46, 0.68) | NS | NS |
| | Day 30 | 0.64* | (0.52, 0.76) | 0.58 | (0.47, 0.69) | NS | NS |
| Eosin/Granulocytes | Baseline | 3.46 | (2.51, 4.78) | 3.50 | (2.61, 4.70) | — | — |
| (10⁹/L) | Day 3 | 3.98 | (2.89, 5.50) | 3.28 | (2.45, 4.41) | NS | NS |
| 1.2-6.8 | Day 14 | 3.80 | (2.75, 5.24) | 3.79 | (2.83, 5.09) | NS | NS |
| | Day 30 | 3.62 | (2.62, 5.00) | 3.52 | (2.63, 4.73) | NS | NS |
| Platelets (10⁹/L) | Baseline | 218 | (180, 255) | 186 | (151, 220) | — | — |
| 150-500 | Day 3 | 201 | (164, 239) | 188 | (154, 222) | NS | NS |
| | Day 14 | 211 | (174, 249) | 173 | (139, 208) | NS | NS |
| | Day 30 | 199 | (162, 237) | 159 | (124, 193) | NS | NS |
| MPV (mm³) | Baseline | 7.35 | (6.54, 8.16) | 7.77 | (7.03, 8.50) | — | — |
| 6.7-11.1 | Day 3 | 7.30 | (6.49, 8.11) | 8.07 | (7.33, 8.80) | NS | NS |
| | Day 14 | 7.34 | (6.53, 8.15) | 8.52* | (7.79, 9.26) | <0.001 | <0.001 |
| | Day 30 | 7.31 | (6.50, 8.12) | 8.63* | (7.90, 9.37) | <0.001 | <0.001 |

Blood total hemoglobin concentration tended to decrease from baseline in the dogs fed the test diets, significantly on day 30, reflecting the decrease in the number of hemoglobin-containing red blood cells. Mean red blood cell hemoglobin (MCH) and mean red blood cell hemoglobin concentration (MCHC) tended to increase, significantly from baseline for MCHC on all sampling days following diet change in test dogs. Neither MCH nor MCHC values were outside the respective lower or upper limits of the reference ranges (Table 2). White blood cell counts were within or close to their respective physiological ranges, despite some transient, yet statistically significant changes (Table 2). Mean platelet volume (MPV) increased compared to baseline in the test group throughout the feeding trial, significantly so on days 14 and 30, but also remained within the reference range.

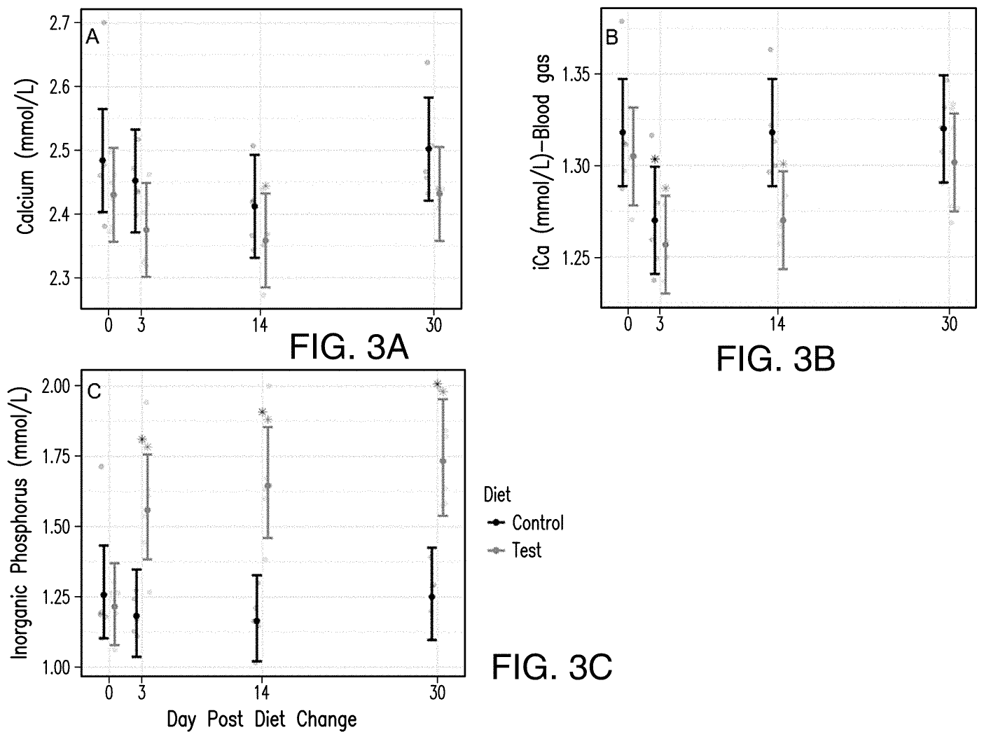

Additional biomarkers such as inorganic molecules and ions were also analyzed. Plasma calcium (Ca) and whole blood ionized calcium (iCa) levels (FIGS. 3A and 3B, respectively) were transiently and significantly decreased in dogs fed the test diet on days 3 and 14, with half the dogs exhibiting plasma calcium levels below the lower limit of the reference range at those time points. Similar results were observed for zinc, although a significant reduction was only observed on day 14, and copper, which showed numerical reductions but without reaching significance (Table 3).

TABLE 3
Mean plasma clinical biochemistry values with 95% confidence intervals for each diet group (Control,
n = 5; Test, n = 6) and sampling time (baseline and 3, 14 and 30 days), as well as results
of the statistical analysis (p-values). Lab reference ranges for each parameter are provided in italics.

| | Sampling | Control | | Test | | Statistical analysis | |
|---|---|---|---|---|---|---|---|
| Parameter | time | Mean | 95% CI | Mean | 95% CI | Diff. within | Diff. bet- |
| Total protein (g/L) | Baseline | 56.1 | (54.0, 58.2) | 57.4 | (55.5, 59.4) | — | — |
| 54.9-75.3 | Day 3 | 55.9 | (53.8, 58.1) | 56.2 | (54.3, 58.2) | NS | NS |
| | Day 14 | 56.6 | (54.5, 58.8) | 56.3 | (54.4, 58.3) | NS | NS |
| | Day 30 | 57.5 | (55.4, 59.6) | 56.8 | (54.8, 58.7) | NS | NS |
| Albumin (g/L) | Baseline | 29.7 | (27.9, 31.5) | 29.7 | (28.0, 31.3) | — | — |
| 26.3-38.2 | Day 3 | 29.7 | (27.9, 31.5) | 29.3 | (27.7, 31.0) | NS | NS |
| | Day 14 | 30.2 | (28.4, 32.0) | 29.4 | (27.8, 31.1) | NS | NS |
| | Day 30 | 30.9* | (29.1, 32.7) | 29.9 | (28.2, 31.5) | NS | NS |
| Globulin (g/L) | Baseline | 26.4 | (24.8, 28.1) | 27.7 | (26.2, 29.3) | — | — |
| 23.4-42.2 | Day 3 | 26.2 | (24.5, 27.9) | 26.9 | (25.3, 28.4) | NS | NS |
| | Day 14 | 26.4 | (24.8, 28.1) | 26.9 | (25.4, 28.4) | NS | NS |
| | Day 30 | 26.6 | (24.9, 28.3) | 26.9 | (25.3, 28.4) | NS | NS |
| Albumin:Globulin ratio | Baseline | 1.13 | (1.02, 1.23) | 1.07 | (0.98, 1.17) | — | — |
| 0.7-1.4 | Day 3 | 1.14 | (1.03, 1.25) | 1.10 | (1.00, 1.20) | NS | NS |
| | Day 14 | 1.15 | (1.04, 1.25) | 1.10 | (1.00, 1.20) | NS | NS |
| | Day 30 | 1.17 | (1.06, 1.27) | 1.12 | (1.02, 1.22) | NS | NS |
| ALAT (U/L) | Baseline | 36.6 | (26.4, 50.6) | 44.5 | (33.1, 59.8) | — | — |
| 19.8-124 | Day 3 | 45.1 | (32.6, 62.5) | 59.9 | (44.5, 80.6) | NS | NS |
| | Day 14 | 47.7 | (34.5, 66.0) | 52.0 | (38.7, 70.0) | NS | NS |
| | Day 30 | 49.9 | (36.1, 69.0) | 48.9 | (36.3, 65.7) | NS | NS |
| ASAT (U/L) | Baseline | 31.9 | (24.7, 41.3) | 39.1 | (30.9, 49.4) | — | — |
| 14-41 | Day 3 | 39.2* | (30.4, 50.7) | 43.0 | (34.1, 54.4) | NS | NS |
| | Day 14 | 41.2* | (31.8, 53.2) | 50.1* | (39.6, 63.3) | <0.001 | NS |
| | Day 30 | 37.3* | (28.9, 48.2) | 44.2 | (35.0, 55.9) | NS | NS |
| Alk. Phosphatase | Baseline | 41.0 | (28.9, 53.1) | 40.7 | (29.6, 51.7) | — | — |
| (U/L) | Day 3 | 48.2 | (36.1, 60.3) | 53.8* | (42.8, 64.9) | 0.002 | NS |
| <130 | Day 14 | 46.4 | (34.3, 58.5) | 58.2* | (47.1, 69.2) | <0.001 | NS |
| | Day 30 | 44.8 | (32.7, 56.9) | 52.8* | (41.8, 63.9) | 0.004 | NS |
| NTproBNP (pmol/L) | Baseline | 738 | (335, 1639) | 521 | (236, 1150) | — | — |
| <900 | Day 30 | 549 | (249, 1210) | 640 | (275, 1490) | NS | NS |
| Creatinine (μmol/L) | Baseline | 90.7 | (79.3, 104) | 87.8 | (77.7, 99.2) | — | — |
| 44-133 | Day 3 | 85.9 | (75.1, 98.3) | 77.7* | (68.7, 87.8) | <0.001 | NS |
| | Day 14 | 91.6 | (80.1, 105.0) | 82.8 | (73.3, 93.6) | NS | NS |
| | Day 30 | 94.1 | (82.3, 108.0) | 87.1 | (77.1, 98.5) | NS | NS |
| Urea (mmol/L) | Baseline | 5.50 | (4.41, 6.59) | 5.56 | (4.56, 6.56) | — | — |
| 3.1-10.1 | Day 3 | 5.14 | (4.05, 6.24) | 4.76 | (3.77, 5.76) | NS | NS |
| | Day 14 | 5.64 | (4.55, 6.74) | 4.71 | (3.71, 5.70) | NS | NS |
| | Day 30 | 5.61 | (4.51, 6.70) | 5.09 | (4.09, 6.08) | NS | NS |
| Cholesterol (mmol/L) | Baseline | 4.25 | (3.46, 5.22) | 4.68 | (3.88, 5.64) | — | — |
| 3.20-6.20 | Day 3 | 4.11 | (3.35, 5.04) | 4.21* | (3.49, 5.08) | 0.005 | NS |
| | Day 14 | 4.05 | (3.30, 4.98) | 4.02* | (3.33, 4.84) | <0.001 | NS |
| | Day 30 | 4.15 | (3.38, 5.10) | 4.14* | (3.43, 5.00) | <0.001 | NS |
| Triglycerides | Baseline | 0.61 | (0.48, 0.77) | 0.49 | (0.39, 0.61) | — | — |
| (mmol/L) | Day 3 | 0.59 | (0.46, 0.75) | 0.47 | (0.38, 0.59) | NS | NS |
| 0.30-1.20 | Day 14 | 0.65 | (0.51, 0.83) | 0.51 | (0.41, 0.64) | NS | NS |
| | Day 30 | 0.68 | (0.54, 0.86) | 0.51 | (0.41, 0.63) | NS | NS |
| Glucose (mmol/L) | Baseline | 5.60 | (5.12, 6.09) | 5.34 | (4.89, 5.78) | — | — |
| 3.6-7.0 | Day 3 | 5.50 | (5.01, 5.98) | 5.19 | (4.75, 5.63) | NS | NS |
| | Day 14 | 5.42 | (4.94, 5.90) | 4.93 | (4.49, 5.37) | NS | NS |
| | Day 30 | 5.65 | (5.17, 6.14) | 4.82* | (4.38, 5.26) | 0.017 | NS |
| Thiamine (pg/L) | Baseline | 59.8 | (47.3, 72.3) | 48.7 | (37.3, 60.1) | — | — |
| 46-112 | Day 3 | 64.8 | (52.3, 77.3) | 53.0 | (41.6, 64.4) | NS | NS |
| | Day 14 | 69.8* | (57.3, 82.3) | 56.5* | (45.1, 67.9) | 0.011 | NS |
| | Day 30 | 65.0 | (52.5, 77.5) | 51.8 | (40.4, 63.2) | NS | NS |
| Vitamin E (mg/L) | Baseline | 24.0 | (19.4, 29.6) | 26.9 | (22.2, 32.6) | — | — |
| 5-50 | Day 3 | 22.7 | (18.4, 28.1) | 21.2* | (17.5, 25.7) | 0.007 | NS |
| | Day 14 | 23.3 | (18.9, 28.8) | 18.7* | (15.4, 22.6) | <0.001 | 0.011 |
| | Day 30 | 22.5 | (18.2, 27.8) | 20.4* | (16.9, 24.8) | 0.001 | NS |
| Calcium (mmol/L) | Baseline | 2.48 | (2.40, 2.56) | 2.43 | (2.36, 2.50) | — | — |
| 2.36-2.84 | Day 3 | 2.45 | (2.37, 2.53) | 2.38 | (2.30, 2.45) | NS | NS |
| | Day 14 | 2.41 | (2.33, 2.49) | 2.36* | (2.28, 2.43) | 0.043 | NS |
| | Day 30 | 2.50 | (2.42, 2.58) | 2.43 | (2.36, 2.51) | NS | NS |

On day 30, however, calcium, ionized calcium, zinc and copper levels were the same as or similar to baseline. Notably, plasma inorganic phosphorus levels (FIG. 3C), on the other hand, were significantly increased in dogs fed the test diet compared to baseline, and significantly more than control-fed dogs, at all three sampling times after diet change. Half the dogs exhibited P levels at or above the upper limit of the reference range at days 14 and 30, and therefore considered hyperphosphataemic.

Additional biochemical markers such as albumin, globulin and other liver enzymes. In particular, total protein, albumin and globulin levels were not affected by the test diet. Liver enzymes alanine aminotransferase (ALAT) and aspartate aminotransferase (ASAT) were increased in all dogs, significantly for ASAT on all sampling days for the control group dogs and at day 14 in the test dogs. ASAT levels were also often above the upper limit of the reference range in dogs on both diet groups, with all test group dogs exhibiting values above 41 U/L at day 14. However, no differences between diets were observed and the values were decreasing in most dogs in both diet groups on day 30. Alkaline phosphatase levels were also increased from baseline at all sampling times, but only in the test group dogs. The heart-specific enzyme NTpro BNP was not significantly affected by diet, although one dog in each diet group exhibited levels above 900 μmol/L.

The levels of carnitine metabolites were also analyzed. Table 4 summarizes serum specific acylcarnitine levels. Transitioning dogs onto the test diet caused a significant increase in carnitine and acetylcarnitine in serum on days 3, 14 and 30 compared to baseline. Significant differences were also observed in test diet for both carnitine and acetylcarnitine. Notably, no significant effects of either diet were observed for propionylcarnitine, isovalerylcarnitine, tetradecenoylcarnitine, hexadexanoylcarnitine or octadecanoylcarnitine.

TABLE 4
Mean serum specific acylcarnitines (in μM) with 95% confidence intervals for each diet group
(Control, n = 5; Test, n = 6) and sampling time (baseline and 3, 14 and 30 days), as
well as results of the statistical analysis (p-values). Parameters are measured in samples from fasted dogs.

| | Sampling | Control | | Test | | Statistical analysis | |
|---|---|---|---|---|---|---|---|
| Parameter | time | Mean | 95% CI | Mean | 95% CI | Diff. within | Diff. |
| Carnitine | Baseline | 17.8 | (11.6, 24) | 16.2 | (10.5, 21.8) | — | — |
| | Day 3 | 17.4 | (11.2, 23.6) | 21.2* | (15.5, 26.8) | <0.001 | 0.031 |
| | Day 14 | 18.2 | (12, 24.4) | 22.3* | (16.7, 28) | <0.001 | 0.017 |
| | Day 30 | 18.8 | (12.6, 25) | 22.5* | (16.9, 28.1) | <0.001 | 0.034 |
| Acetylcarnitine | Baseline | 1.49 | (1.06, 2.07) | 1.32 | (0.972, 1.79) | — | — |
| | Day 3 | 1.58 | (1.13, 2.21) | 1.98* | (1.46, 2.69) | <0.001 | 0.021 |
| | Day 14 | 1.65 | (1.18, 2.31) | 2.01* | (1.48, 2.72) | <0.001 | 0.047 |
| | Day 30 | 1.73 | (1.24, 2.41) | 2.12* | (1.56, 2.88) | <0.001 | 0.033 |
| Propionylcarnitine | Baseline | 0.140 | (0.07, 0.21) | 0.109 | (0.045, 0.173) | — | — |
| | Day 3 | 0.128 | (0.0584, 0.198) | 0.142 | (0.0780, 0.205) | NS | NS |
| | Day 14 | 0.111 | (0.0412, 0.181) | 0.148 | (0.0840, 0.211) | NS | NS |
| | Day 30 | 0.149 | (0.0790, 0.219) | 0.160 | (0.0963, 0.224) | NS | NS |
| Isovalerylcarnitine | Baseline | 0.172 | (0.0842, 0.259) | 0.128 | (0.0482, 0.208) | — | — |
| | Day 3 | 0.184 | (0.0964, 0.272) | 0.136 | (0.0558, 0.216) | NS | NS |
| | Day 14 | 0.164 | (0.0764, 0.252) | 0.147 | (0.0667, 0.227) | NS | NS |
| | Day 30 | 0.188 | (0.100, 0.276) | 0.153 | (0.0733, 0.233) | NS | NS |
| Tetradecenoyl- | Baseline | 0.116 | (0.0835, 0.149) | 0.118 | (0.0886, 0.148) | — | — |
| carnitine | Day 3 | 0.112 | (0.0799, 0.145) | 0.111 | (0.0808, 0.140) | NS | NS |
| | Day 14 | 0.113 | (0.0805, 0.146) | 0.099 | (0.0693, 0.129) | NS | NS |
| | Day 30 | 0.116 | (0.0839, 0.149) | 0.090 | (0.0601, 0.120) | NS | NS |
| Hexadexanoyl- | Baseline | 0.071 | (0.0547, 0.0920) | 0.0626 | (0.0493, 0.0793) | — | — |
| carnitine | Day 3 | 0.0744 | (0.0574, 0.0964) | 0.0614 | (0.0484, 0.0778) | NS | NS |
| | Day 14 | 0.0712 | (0.0549, 0.0923) | 0.0616 | (0.0486, 0.0781) | NS | NS |
| | Day 30 | 0.0745 | (0.0574, 0.0965) | 0.0608 | (0.0479, 0.0770) | NS | NS |
| Octadecanoyl- | Baseline | 0.0402 | (0.0268, 0.0536) | 0.0347 | (0.0224, 0.0469) | — | — |
| carnitine | Day 3 | 0.0350 | (0.0216, 0.0484) | 0.0343 | (0.0221, 0.0466) | NS | NS |
| | Day 14 | 0.0356 | (0.0222, 0.0490) | 0.0350 | (0.0227, 0.0473) | NS | NS |
| | Day 30 | 0.0364 | (0.0230, 0.0498) | 0.0353 | (0.0231, 0.0476) | NS | NS |

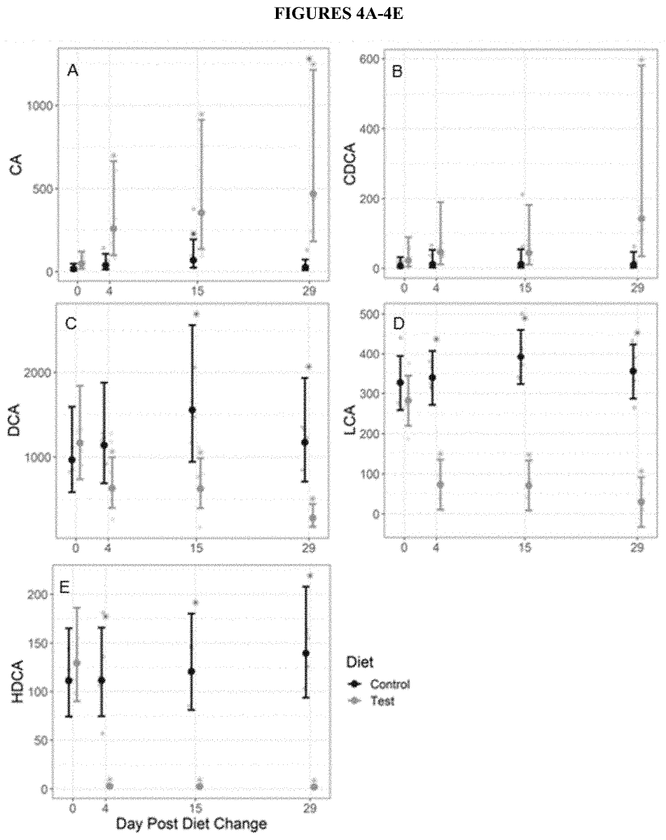

Fecal bile acid profiling was also performed and is summarized in FIGS. 4A-4E. Significant differences from baseline were detected within and between diets. Test diet-feeding caused increased fecal concentrations of primary bile acids cholic acid (CA), chenodeoxycholic acid (CDCA) and MCAα+13, and primary bile salt concentration TMCAα+13 in feces compared to baseline. When total fecal losses were calculated using quantified feces outputs, test diet-fed dogs' average fecal losses at day 30 were 17.8, 6.5, 9 and 36-fold higher, respectively, than their baseline losses. On the other hand, secondary bile acid/salt levels and losses were generally reduced. Thus, for the primary bile acids/salts an overall increase in fecal bile acid losses was observed, while for secondary bile acids/salts, fecal losses or production via microbial metabolism were decreased.

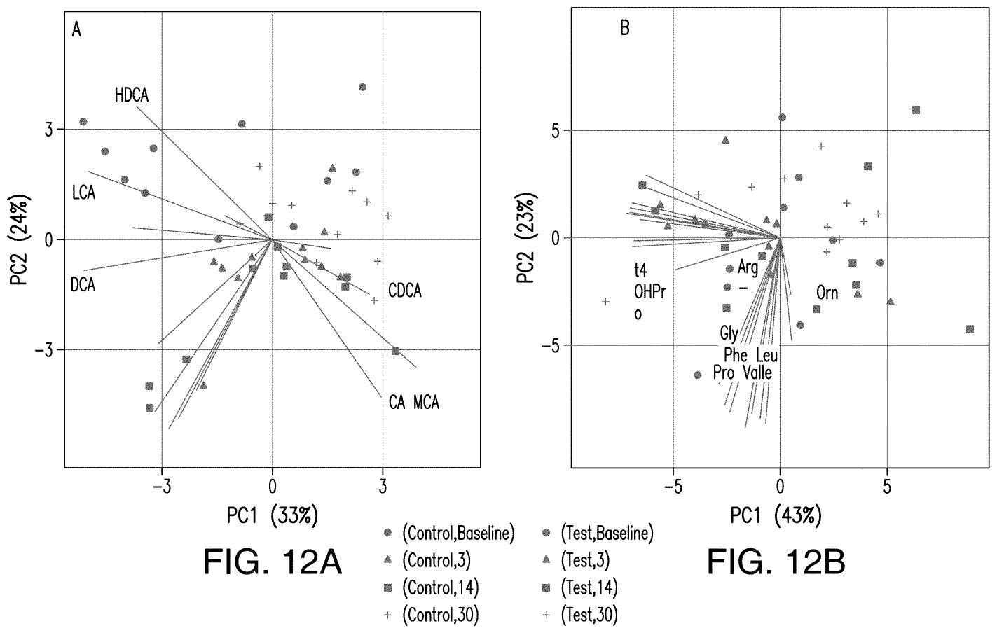

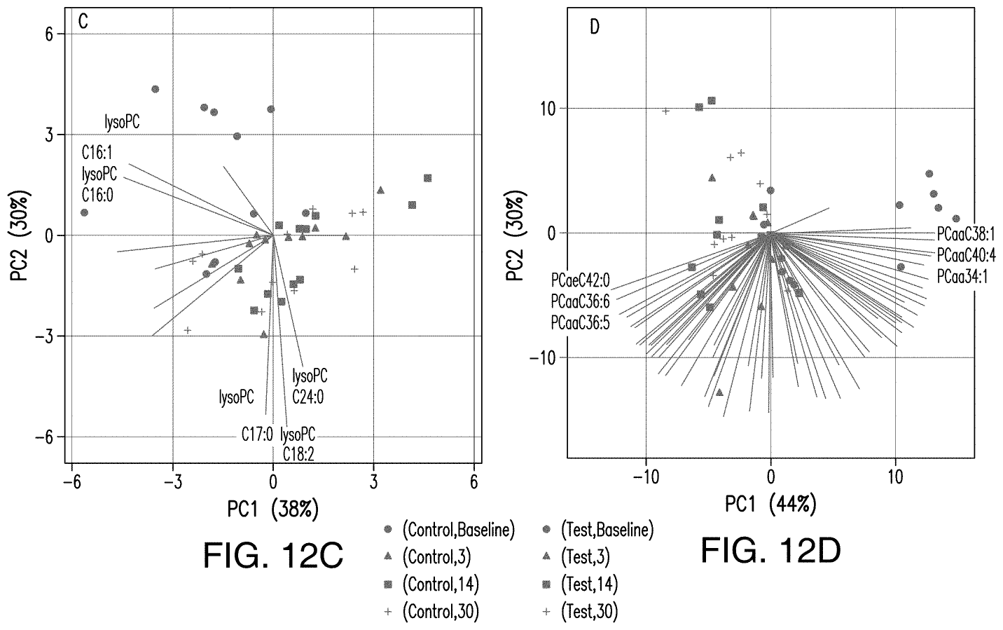

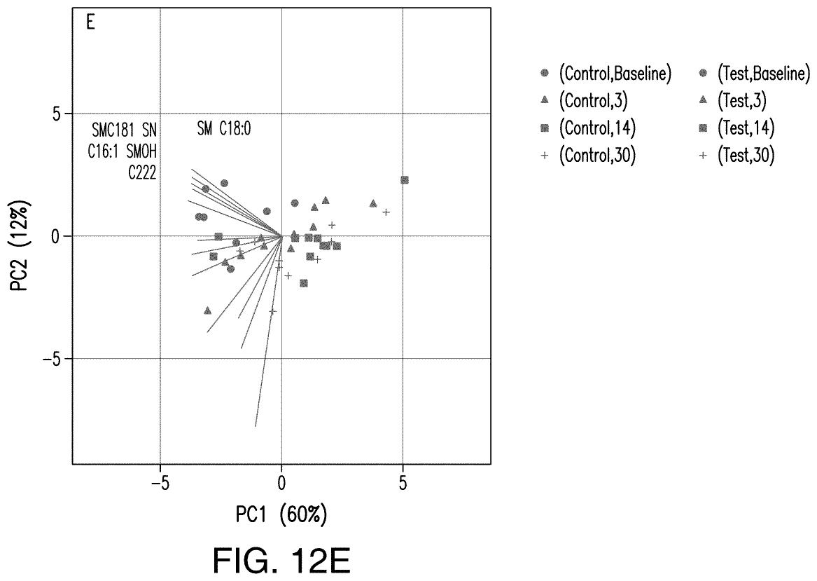

In the present example, serum metabolomics was also analyzed. The obtained data are summarized in FIGS. 12A-12E and show the multivariate analyses performed on these and the fecal bile acids, indicating the associations detected between the various metabolites, diet group and sampling times. FIG. 12A indicates the increases in primary bile acids CA, total MCA and CDCA with increased exposure to the legume-rich test diet, whereas secondary bile acids DCA, LCA and HDCA were higher at baseline and decreased with test diet exposure, as also indicated in FIGS. 4A-4E.

For the serum amino acids and biogenic amines (FIG. 12B), arginine and ornithine, represented by short lines, increased in dogs fed the test diet, whereas glycine, phenylalanine, proline and its metabolite trans-4-hydroxy-proline (t4-OH-Pro), and the branch-chained amino acids leucine, isoleucine and valine were higher at baseline. As indicated in FIG. 12B, plasma and serum levels of methionine, cystine and lysine were not affected by diet. Serum lysophosphatidylcholines with shorter chain residues, specifically lysoPC C16:0 and C16:1 were higher at baseline, while those with longer chained residues C17:0, C18:2 and C24:0 were increased when dogs were fed the test diet (FIG. 12C). As indicated in FIG. 12D, many phosphatidylcholines measured in serum were significantly affected by the test diet. However, no obvious trends in responses as affected by binding type, length or degree of saturation of the different residue chains were observed. Serum levels of hydroxy-sphingomyelins and sphingomyelins affected by diet (e.g., SM(OH) C22:2 and SM C16:1, C18:0 and C18:1) and were all reduced when dogs were transitioned onto the test diet, explaining the respective lines pointing toward the cluster of baseline sampling time for the test group dogs and the higher baseline values (FIG. 12E).

### Discussion

The data from this pilot study of the present example indicate that feeding adult dogs, e.g., Labrador retriever dogs, a diet containing a combination of peas and red and green lentils (20% inclusion of each) for 30 days caused reduced nutrient digestibility, a rapid reduction in red blood cell counts, hyperphosphatemia, and increased fecal losses of primary bile acids, with possible implications on sterol metabolism and taurine status, as indicated by reduced urinary losses of taurine. Reports from humans and rodents indicate that these data have the potential to explain pathophysiological processes in the heart muscle. Interrogation of medical records from dogs diagnosed with DCM, as well as more refined and longer-term feeding trials, is needed to investigate whether the findings from this 30-day study are transient or self-limiting, or whether they are indeed newly discovered causes of the diet-induced, canine DCM cases recently highlighted by the FDA.

The anti-nutritional factors and/or fiber present in the legume-rich test diet did appear to impact nutrient digestibility. If one or more nutrient deficiencies specifically affect heart health and explain the development of DCM in dogs fed such diets over long periods.

The increasing severity of the clinically apparent normocytic anemia observed in the test group dogs from day 3 of the trial (FIGS. 2A-2B) was not caused by low iron levels in the diet, as iron was higher in the test compared to the control diet (Table 1). However, decreased iron bioavailability for hemoglobin production can become an issue over time in dogs fed legume-rich diets, due to the high anti-nutrient and fiber content, as indicated by reduced nutrient digestibility. Yet it is unlikely to have been the cause of anemia in the current, short term trial as body stores of iron would be mobilized first, as also indicated by the stable MCH and MCHC levels (Table 2). Although anemia has not been cited as a cause of DCM in dogs, reduced red blood cell numbers will reduce efficiency in oxygen transport, which can put undue strain on the heart muscle when increased blood volumes are required to meet peripheral tissue needs for oxygen. At the very least, anemia can exacerbate signs and complicate recovery in DCM patients if not discovered and treated.

Anti-nutritional factors (ANFs) present in the test diet cannot be ruled out as causes of the anemia. Saponins present in some legumes are known to cause hemolysis by disrupting cell membranes due to their amphipathic nature. Legume lectins can agglutinate cells, including red blood cells, by binding to carbohydrate moieties on the cell surface. When cyanide is released from cyanogenic compounds in flaxseed, it can also cause red blood cell death by halting cellular respiration.

The increasing number of individuals affected and increasing severity of hyperphosphataemia (FIG. 3C) observed in the test diet-fed dogs from day 3 of the trial can be a sign of increased phosphate availability for absorption. Changes in mineral interactions in the intestine, possibly caused by high phytate and fiber in legume-rich diets, explains greater availability of phosphate for absorption. This is due because 1) the high plasma phosphate was most likely not caused by dietary phosphate levels, as the test diet contained about 1.4 g/Mcal (0.49%) compared to 2.5 g/Mcal (0.91%) in the control diet (Table 1), and 2) transient disruptions in plasma levels of Ca, iCa, Mg, K, Zn and Na:K ratio were also evident (Table 2). A hormonal response, such as secretion of parathyroid hormone (PTH), fibroblast growth factor 23 (FGF-23) and/or vitamin D metabolites, elicited by the low plasma calcium/ionized calcium and/or high plasma phosphate would be expected in an attempt to restore homeostasis, as indicated by more or less restored levels of most minerals at day 30, with the notable exception of phosphate. Despite the hyperphosphatemia, no signs of renal distress were observed in the test group dogs, with serum creatinine and urea, and urine specific gravity within reference ranges.

An alternative cause of hyperphosphataemia in dogs is hemolytic anemia, the disruption of red blood cells causing intracellular phosphate to leak into the plasma. The analyses of ingredients and diets for ANF content/activity provides insight into anemia as a cause of hyperphosphataemia.

Liver function appeared to be minimally affected by diet as assessed by the bile acid stimulation test results, but increases in plasma liver enzyme levels of ASAT and possibly alkaline phosphatase (Table 3) suggest it can have been transiently affected in the test group dogs by the change to the legume-rich diet. Thus, liver damage was not suspected to have been caused by the short term feeding of the legume-rich test diet. However, the concomitant decrease in plasma cholesterol (Table 3), increased postprandial serum bile acids, and increased serum primary bile salts can indicate that hepatic bile acid synthesis was increased following diet transition, possibly in response to increased fecal losses of primary bile acids putting a strain on liver function and causing changes in liver enzyme levels in plasma. Alkaline phosphatase is present in liver cells and can therefore be a marker for liver health, but it is also an enzyme present in bony tissue and involved in the bone mineral turnover. Thus, the increased plasma levels can also be explained by changes in mineral homeostasis, as discussed above.

Taurine levels in whole blood and plasma were not reduced in the test group dogs, confirming observations in dogs suffering from DCM in the recent outbreak (Freeman et al. 2018). Taurine levels were transiently increased on day 14, which was most likely due to up-regulated biosynthesis from methionine and cysteine. This is indicated by increases in methionine cycle metabolite DMG, apparently at the expense of sarcosine synthesis (FIGS. 3A-3C), and decreases in plasma cysteine levels, significant at day 30. However, decreased taurine levels in pooled urine indicate that biosynthesis can only marginally meet taurine requirements by day 30, and increased fecal losses of bile acids and salts can eventually detrimentally affect taurine status. This can have been compounded by dietary methionine and cysteine levels only marginally meeting recommended allowances and are therefore largely required for protein synthesis, as was the case in for the test diets (Table 1). The increased need for taurine to meet increased bile salt synthesis as a response to increased fecal bile acid/salt losses, can therefore have contributed to the decreases in urinary excretion of taurine as indicated by decreased taurine:creatinine ratio and pooled urine values. Therefore, urinary taurine levels when corrected for creatinine, can be a more sensitive measure of taurine depletion in dogs than whole blood or plasma taurine.

Interestingly, serum and fecal secondary bile acids and salts were generally reduced in the test compared to control group dogs, sometimes by an order of magnitude. This indicates that the microbial metabolism of primary bile acids and salts is decreased in dogs fed the legume-rich test diets.

Among other nutrients and substances that have been implicated in cardiomyopathies across species, vitamin E deficiency cannot be ruled out as serum levels decreased throughout the study in the test group dogs. However, vitamin E levels in the test diet were lower than in the control, and this response can therefore be a reflection of decreased supply. Plasma vitamin B1 and serum carnitine levels tended to increase in the test diet group, with higher levels in the test diet a likely cause of the plasma vitamin B1 increases. In a recent review, Albakri (2019) also indicated that vitamin B3 (niacin) and Coenzyme Q10 (ubiquinone) deficiencies can be causes of cardiomyopathies in humans. While these were not investigated in the present example, they deserve scrutiny in future trials. Furthermore, changes in secondary bile acid, glycine, branched-chain amino acid, t4-OH-proline, biogenic amine, lysophosphatidylcholine, phosphatidylcholine and sphingomyelin levels and metabolism, as well as lipotoxicity involvement have been implicated in cardiovascular disease and/or cardiomyopathies in humans (Park & Goldberg 2012; Lu et al. 2017; Mueller-Hennessen et al. 2017; Kikas et al. 2018; Vasavan et al. 2018; Law et al. 2019), but have not been investigated in canine DCM so far. However, clues for such associations can be present among the serum metabolomics data (FIGS. 12A-12E) from the present example.

### CONCLUSION

The data of the present example indicate that the legume-rich test diet containing peas, lentils and a relatively high level of flaxseed can cause reduced nutrient digestibility, normocytic anemia and hyperphosphatemia, as well as possible disturbances in taurine and sterol metabolism due to increased primary bile acid losses in feces. Metabolomic profiling also indicated further changes in serum secondary bile acids/salts, glycine, branched-chain amino acid, t4-OH-proline, biogenic amine, lysophosphatidylcholine, phosphatidylcholine and sphingomyelin levels. Fecal microbiota profiling and a retrospective interrogation of medical records of dogs with diet-induced DCM can increase the understanding of the responses measured to date.

Treating legume meals with different processing methods (e.g. heat, solvent extraction, fermentation) before extrusion can help identify ANFs involved in responses observed. Example 2

The present example investigates and identifies biomarkers for predicting development of DCM by using a machine learning approach.

To investigate the machine-learning approach for prognostic and diagnostic methods, a total of 39,169 visit records were collected between 2018 and 2020 and the data of 32 features of demographics, blood chemistry and hematology were extracted and used. 38,602 records were obtained from healthy controls and 567 records were obtained from DCM cases. Disease prevalence was assessed at 1.4%. Segmented neutrophils (%) and amylase (AMYL) features were removed and a machine learning (ML) approach was used to fill missing data.

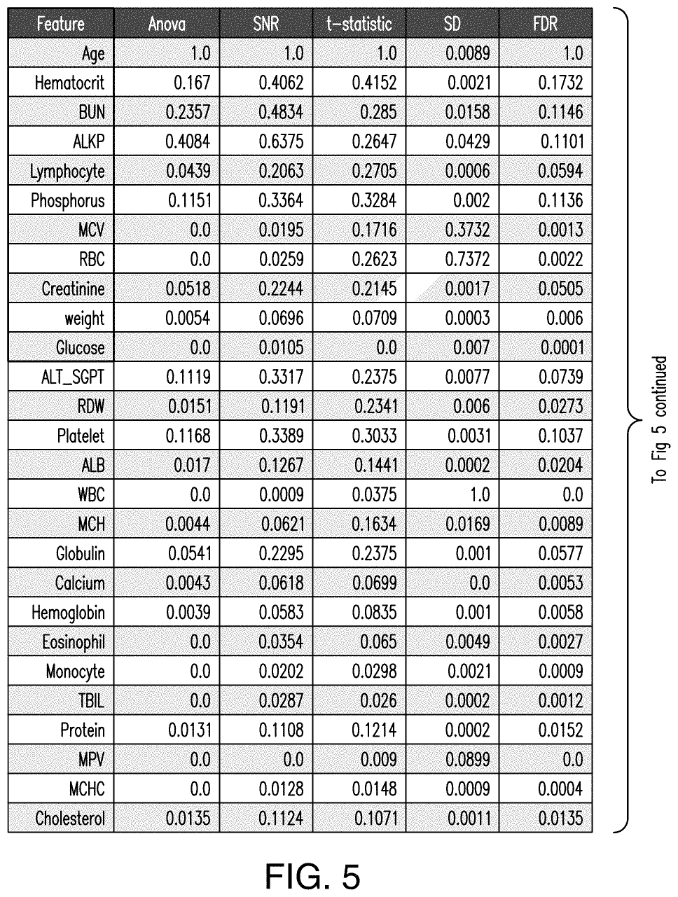

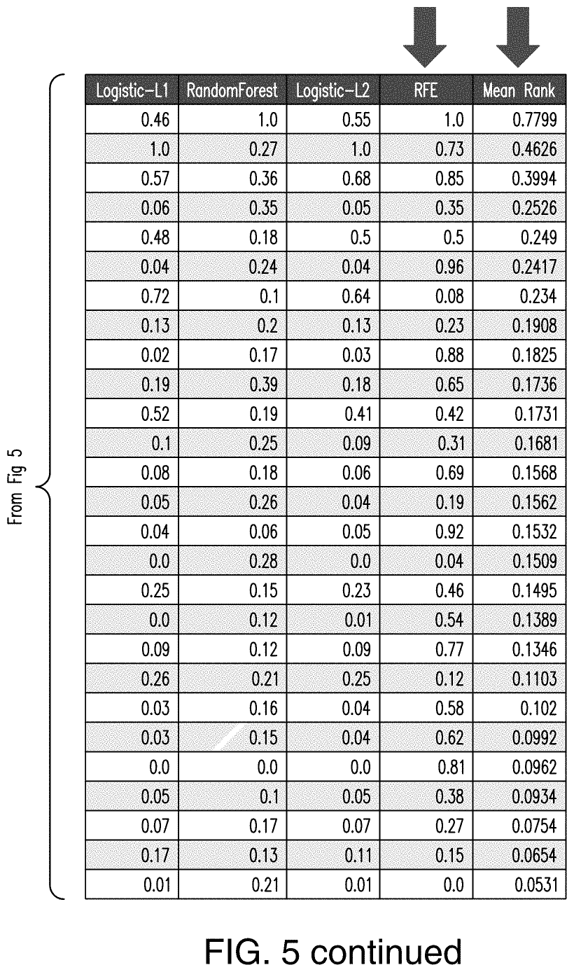

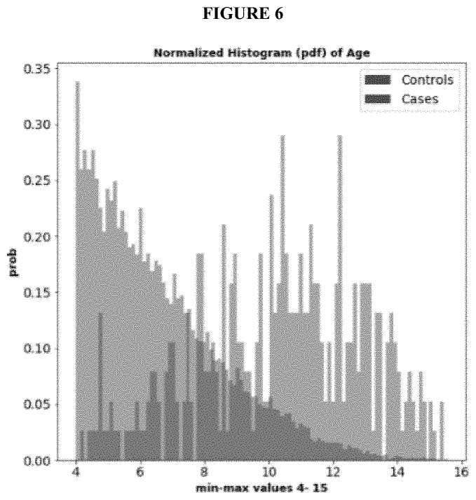

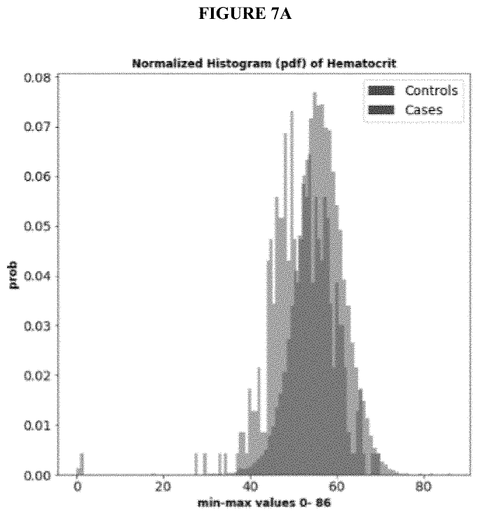

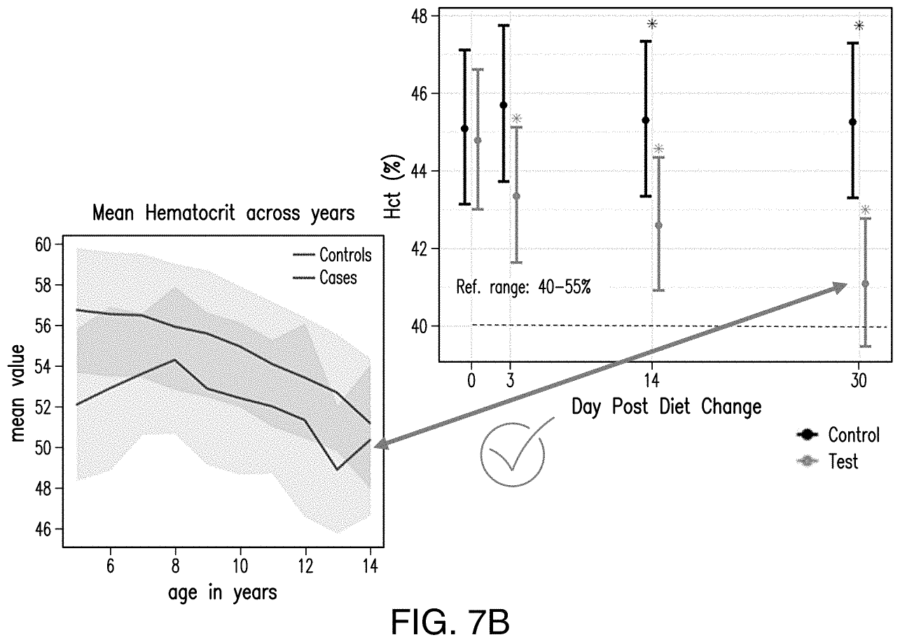

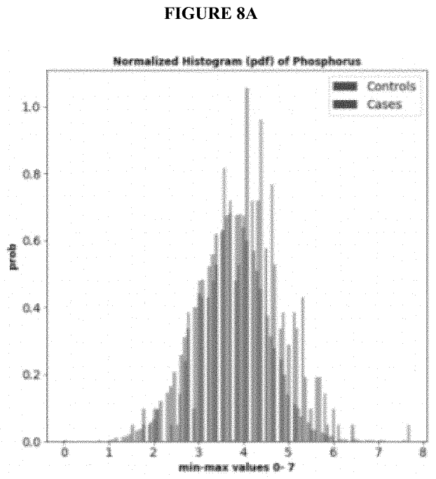

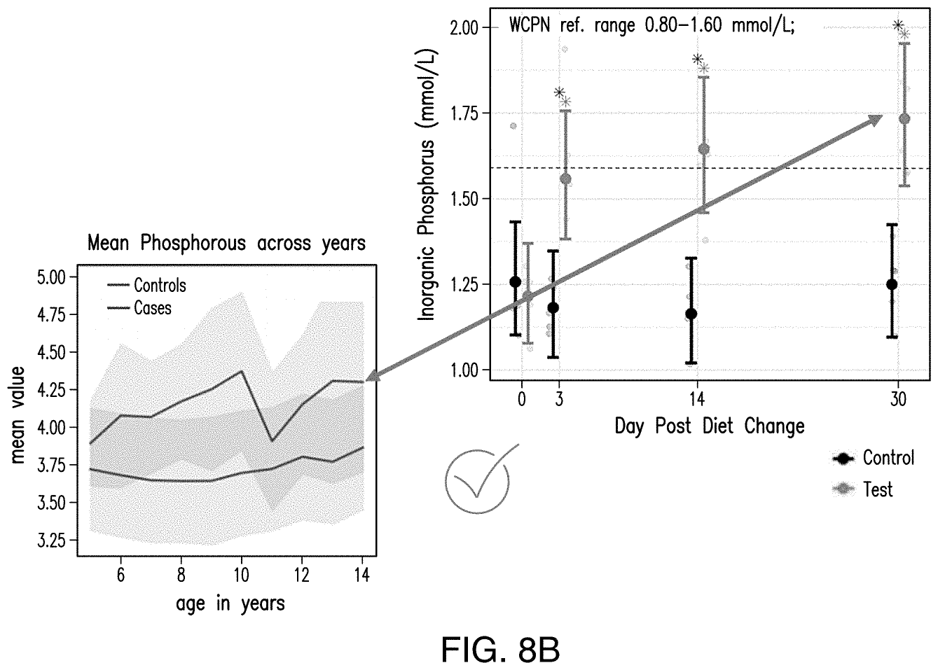

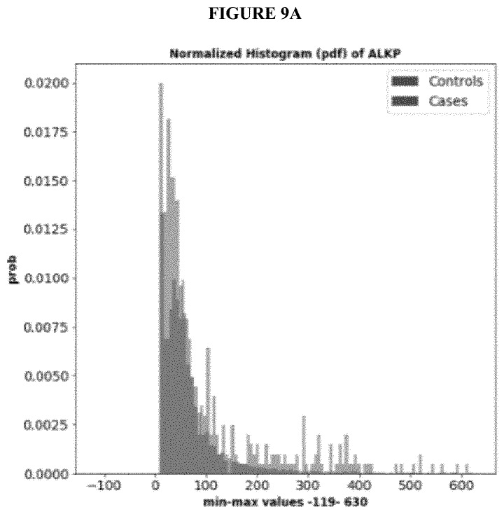

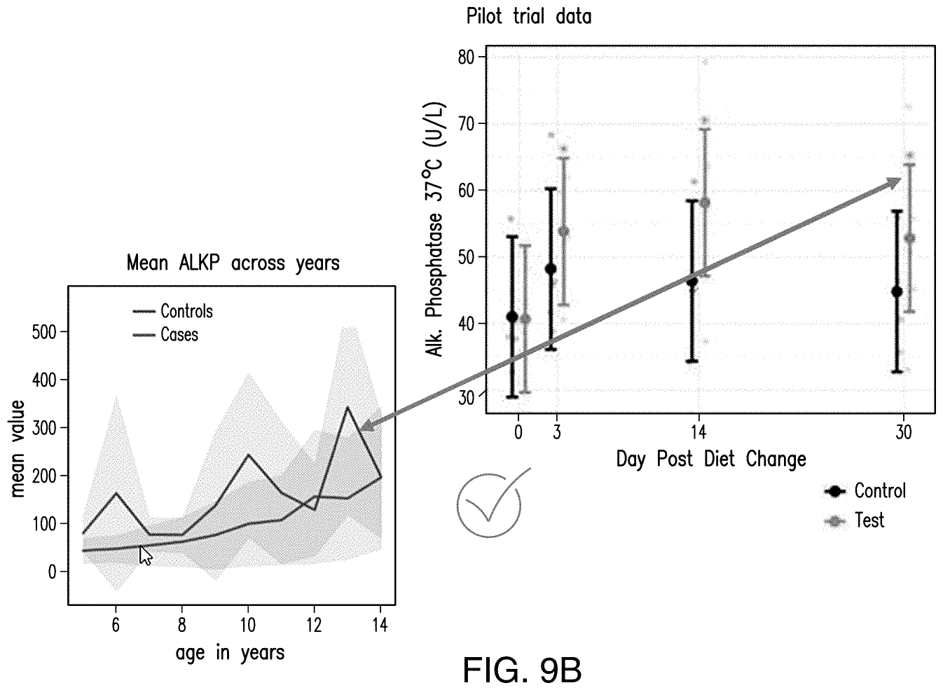

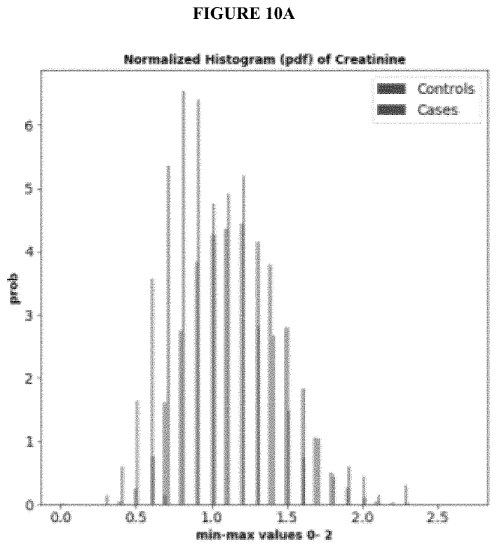

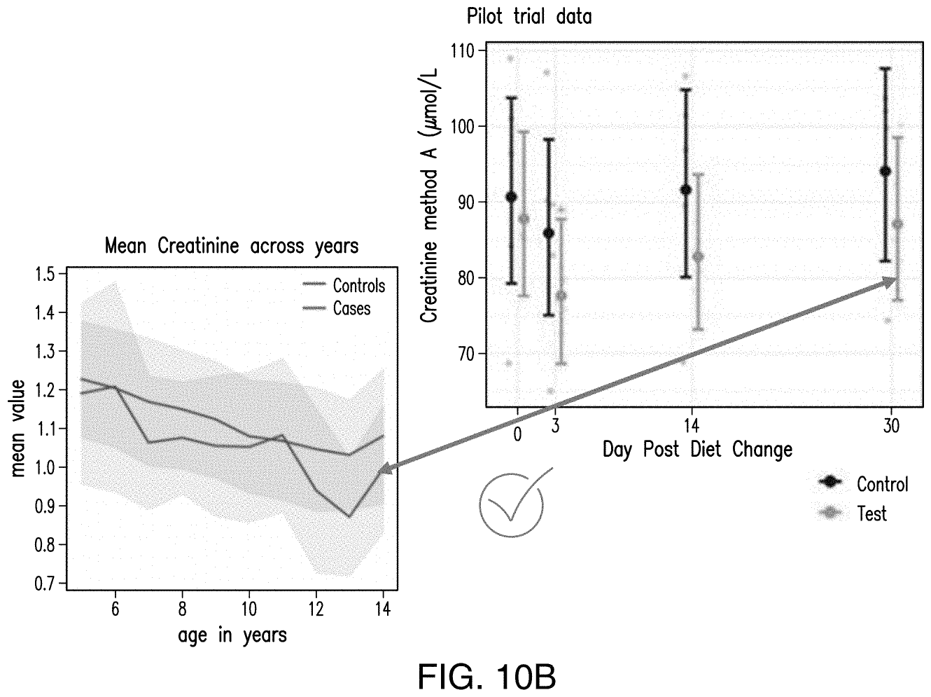

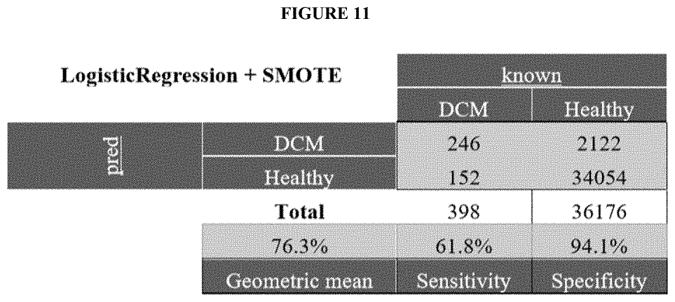

The predictive performance of different features was determined and ranked by importance. As shown in FIG. 5, the following biomarkers ranked at the top: age, hematocrit, BUN, ALKP, lymphocyte number, phosphorus, MCV, RBC, creatinine, weight and glucose. FIG. 6 shows the exploratory data analysis of the probability of developing DCM based on age. Under normal circumstances, the disease is most commonly diagnosed in older dogs. Clinical signs in dogs with diet-induced DCM appeared to take a minimum of 9 months to develop while younger affected dogs have been reported to the FDA. Interestingly, as shown in the exploratory data analysis in FIGS. 7A-10B, hematocrit, phosphorus, alkaline phosphatase and creatinine predicted the development of DCM observed in the clinical study described in Example 1. In particular, a decrease in hematocrit (e.g., <45%, with 40% being the lower end of the physiological range) was a predictor of the development of DCM; an increase in plasma inorganic phosphate (e.g., >1.5 mmol/L, with 1.6 being the upper end of the physiological range); an increase in alkaline phosphatase (e.g., >50 U/L, with 90 U/L being the upper physiological range); and a decrease in creatinine (e.g., <100 μmol/L, with 65 μmol/L being the lower physiological range). Thus, these biomarkers (hematocrit, phosphorus, alkaline phosphatase and creatinine) can be used for prognostic and diagnostic methods to predict and treat DCM.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method of treating dilated cardiomyopathy (DCM) in a dog, the method comprising:
a) measuring, in a sample from the dog, an amount of at least one biomarker; and
b) administering a treatment or a dietary regimen to treat or prevent DCM.

2. The method of claim 1, wherein the at least one biomarker comprises hematocrit, inorganic phosphate, alkaline phosphatase, creatinine, or any combination thereof.

3. The method of claim 2, wherein an amount of the hematocrit is below about 45%.

4. The method of claim 2, wherein an amount of the inorganic phosphate is above about 1.5 mmol/L.

5. The method of claim 2, wherein an amount of the creatinine is below about 100 μmol/L.

6. The method of claim 2, wherein an amount of the alkaline phosphatase is above about 50 U/L.

7. The method of claim 2, wherein the dog is at risk of developing DCM if an amount of hematocrit is below about 45%, an amount of the inorganic phosphate is above about 1.5 mmol/L, an amount of the creatinine is below about 100 μmol/L, and an amount of the alkaline phosphatase is above about 50 U/L.

8. The method of claim 2, wherein the at least one biomarker further comprises a primary bile acid, a primary bile salt, a secondary bile acid, a secondary bile salt, alkaline phosphatase, amylase, total protein, BUN or urea level, phosphorus, calcium, urine protein, potassium, glucose, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine pH level, white blood cell count, or a combination thereof.

9. The method of claim 1, wherein the dietary regimen comprises a low phosphorus diet, a low legume diet, a taurine supplement diet, a carnitine supplement diet, a low protein diet, a low sodium diet, a potassium supplement diet, a polyunsaturated fatty acid supplement diet, a liquid diet, a calcium supplement diet, a regular protein diet, or any combination thereof.

10. The method of claim 9, wherein the dietary regimen comprises a low legume diet, a taurine supplement diet, a carnitine supplement diet, or any combination thereof.

11. A method of preventing or reducing the risk of developing dilated cardiomyopathy (DCM) in a dog, the method comprising:
a) measuring, in a sample from the dog, an amount of at least one biomarker; and
b) administering a treatment or a dietary regimen to treat or prevent DCM.

12. The method of claim 11, wherein the at least one biomarker comprise hematocrit, inorganic phosphate, alkaline phosphatase, creatinine, or any combination thereof.

13. The method of claim 12, wherein an amount of the hematocrit is below about 45%.

14. The method of claim 12, wherein an amount of the inorganic phosphate is above about 1.5 mmol/L.

15. The method of claim 12, wherein an amount of the creatinine is below about 100 μmol/L.

16. The method of claim 12, wherein an amount of the alkaline phosphatase is above about 50 U/L.

17. The method of claim 12, wherein the dog is at risk of developing DCM if an amount of hematocrit is below about 45%, an amount of the inorganic phosphate is above about 1.5 mmol/L, an amount of the creatinine is below about 100 μmol/L, and an amount of the alkaline phosphatase is above about 50 U/L.

18. The method of claim 12, wherein the at least one biomarker further comprises a primary bile acid, a primary bile salt, a secondary bile acid, a secondary bile salt, alkaline phosphatase, amylase, total protein, BUN or urea level, phosphorus, calcium, urine protein, potassium, glucose, hemoglobin, red blood cell (RBC) count, red cell distribution width (RDW), alanine aminotransferase, albumin, bilirubin, chloride, cholesterol, eosinophil, globulin, lymphocyte, monocyte, mean corpuscular hemoglobin (MCH), mean corpuscular hemoglobin concentration (MCHC), mean corpuscular volume (MCV), mean platelet volume (MPV), platelet count, segmented neutrophils, sodium, urine pH level, white blood cell count, or a combination thereof.

19. The method of claim 11, wherein the dietary regimen comprises low legume diet, a taurine supplement diet, a carnitine supplement diet, a low phosphorus diet, a low protein diet, a low sodium diet, a potassium supplement diet, a polyunsaturated fatty acid supplement diet, a liquid diet, a calcium supplement diet, a regular protein diet, or any combination thereof.

20. The method of claim 19, wherein the dietary regimen comprises a low legume diet, a taurine supplement diet, a carnitine supplement diet, or any combination thereof.
